(19) United States
(12) Patent Application Publication
(10) Pub. No.: US 2005/0009022 A1
(43) Pub. Date: Jan. 13, 2005

(54) Method for isolation of independent, parallel chemical micro-reactions using a porous filter

(76) Inventors: Michael P. Weiner, Guilford, CT (US); Said Attiya, Branford, CT (US); Hugh C. Crenshaw, Durham, NC (US); Jonathan M. Rothberg, Guilford, CT (US); Stephen L. Matson, Harvard, MA (US)

Correspondence Address: MINTZ LEVIN COHN FERRIS GLOVSKY & POPEO, 666 THIRD AVENUE, NEW YORK, NY, 10017, US

(21) Appl. No.: 10/482,491

(22) PCT Filed: Jul. 8, 2002
(86) PCT No.: PCT/US02/21579

(30) Foreign Application Priority Data
Jul. 6, 2001 (US) 60303576

(51) Int. Cl. C12Q 1/68; C12M 1/34

(52) U.S. Cl. 435/6; 435/287.2

(57) ABSTRACT

The present invention relates to the field of fluid dynamics. More specifically, this invention relates to methods and apparatus for conducting densely packed, independent chemical reactions in parallel in a substantially two-dimensional array. Accordingly, this invention also focuses on the use of this array for applications such as DNA sequencing, most preferably pyrosequencing, and DNA amplification.

### FIELD OF THE INVENTION

The invention describes method and apparatus for conducting densely packed, independent chemical reactions in parallel in a substantially two-dimensional array comprising a porous filter.

### BACKGROUND OF THE INVENTION

High throughput chemical synthesis and analysis are rapidly growing segments of technology for many areas of human endeavor, especially in the fields of material science, combinatorial chemistry, pharmaceuticals (drug synthesis and testing), and biotechnology (DNA sequencing, genotyping).

Increasing throughput in any such process requires either that individual steps of the process be performed more quickly, with emphasis placed on accelerating rate-limiting steps, or that larger numbers of independent steps be performed in parallel. Examples of approaches for conducting chemical reactions in a high-throughput manner include such techniques as:

Performing a reaction or associated processing step more quickly:

Adding a catalyst

Performing the reaction at higher temperature

Performing larger numbers of independent steps in parallel:

Conducting simultaneous, independent reactions with a multi-reactor system. A common format for conducting parallel reactions at high throughput comprises two-dimensional (2-D) arrays of individual reactor vessels, such as the 96-well or 384-well microtiter plates widely used in molecular biology, cell biology, and other areas. Individual reagents, solvents, catalysts, and the like are added sequentially and/or in parallel to the appropriate wells in these arrays, and multiple reactions subsequently proceed in parallel. Individual wells may be further isolated from adjacent wells and/or from the environment by sealing means (e.g., a tight-fitting cover or adherent plastic sheet) or they may remain open. The base of the wells in such microtiter plates may or may not be provided with filters of various pore sizes. The widespread application of robotics has greatly increased the speed and reliability of reagent addition, supplementary processing steps, and reaction monitoring—thus greatly increasing throughput.

Further increasing the number of microvessels or microreactors incorporated in such 2-D arrays has been a focus of much research, and this has been and is being accomplished by miniaturization. For instance, the numbers of wells that can be molded into plastic microtiter plates has steadily increased in recent years—from 96, to 384, and now to 1536. Efforts to further increase the density of wells are ongoing (e.g. Matsuda and Chung, 1994; Michael et al., 1998; Taylor and Walt, 1998).

Attempts to make arrays of microwells and microvessels for use as microreactors has also been a focal point for development in the areas of microelectromechanical and micromachined systems, applying and leveraging some of the microfabrication techniques originally developed for the microelectronics industry (see Matsuda and Chung, 1994; Rai-Choudhury, 1997; Madou, 1997; Cherukuri et al., 1999; Kane et al. 1999; Anderson et al., 2000; Dannoux et al., 2000; Deng et al., 2000; Zhu et al., 2000; Ehrfeld et al., 2000).

Yet another widely applied approach for conducting miniaturized and independent reactions in parallel involves spatially localizing or immobilizing at least some of the participants in a chemical reaction on a surface, thus creating large 2-D arrays of immobilized reagents. Reagents immobilized in such a manner include chemical reactants, catalysts, other reaction auxiliaries, and adsorbent molecules capable of selectively binding to complementary molecules. (For purposes of this patent specification, the selective binding of one molecule to another—whether reversible or irreversible—will be referred to as a reaction process, and molecules capable of binding in such a manner will be referred to as reactants.) Immobilization may be arranged to take place on any number of substrates, including planar surfaces and/or high-surface-area and sometimes porous support media such as beads or gels. Microarray techniques involving immobilization on planar surfaces have been commercialized for the hybridization of oligonucleotides (e.g. by Affymetrix Inc.) and for target drugs (e.g. by Graffinity AB).

A major obstacle to creating microscopic, discrete centers for localized reactions is that restricting unique reactants and products to a single, desired reaction center is frequently difficult. There are two dimensions to this problem. The first is that “unique” reagents—i.e., reactants and other reaction auxiliaries that are meant to differ from one reaction center to the next—must be dispensed or otherwise deployed to particular reaction centers and not to their nearby neighbors. (Such “unique” reagents are to be distinguished from “common” reagents like solvents, which frequently are meant to be brought into contact with substantially all the reaction centers simultaneously and in parallel.) The second dimension of this problem has to do with restricting reaction products to the vicinity of the reaction center where they were created—i.e., preventing them from traveling to other reaction centers with attendant loss of reaction fidelity.

Focusing first on the problem of directed reagent addition, if the reaction center consists of a discrete microwell—with the microvessel walls (and cover, if provided) designed to prevent fluid contact with adjacent microwells—then delivery of reagents to individual microwells can be difficult, particularly if the wells are especially small. For example, a reactor measuring 100 μm×100 μm×100 μm has a volume of only 1 nanoliter—and this can be considered a relatively large reactor volume in many types of applications. Even so, reagent addition in this case requires that sub-nanoliter volumes be dispensed with a spatial resolution and precision of at least ±50 μM. Furthermore, addition of reagents to multiple wells must be made to take place in parallel, since sequential addition of reagents to at most a few reactors at a time would be prohibitively slow. Schemes for parallel addition of reagents with such fine precision exist, but they entail some added complexity and cost. For example, the use of inkjet print technologies to deliver sub-nanoliter-sized drops to surfaces has been widely explored and developed (Gamble et al, 1999; Hughes et al., 2001; Rosetta, Inc.; Agilent, Inc.) However, evaporation of such small samples remains a significant problem that requires careful humidity control.

If, on the other hand, the reaction centers are brought into contact with a common fluid—e.g., if the microwells all open out onto a common volume of fluid at some point during the reaction or subsequent processing steps—then reaction products (and excess and/or unconverted reactants) originating in one reaction microwell or vessel can travel to and contaminate adjacent reaction microwells. Such cross-contamination of reaction centers can occur (i) via bulk convection of solution containing reactants and products from the vicinity of one well to another, (ii) by diffusion (especially over reasonably short distances) of reactant and/or product species, or (iii) by both processes occurring simultaneously. If the individual chemical compounds that are produced at the discrete reaction centers are themselves the desired objective of the process (e.g., as is the case in combinatorial chemistry), then the yield and ultimate chemical purity of this “library” of discrete compounds will suffer as a result of any reactant and/or product cross-contamination that may occur. If, on the other hand, the reaction process is conducted with the objective of obtaining information of some type—e.g., information as to the sequence or composition of DNA, RNA, or protein molecules—then the integrity, fidelity, and signal-to-noise ratio of that information may be compromised by chemical “cross-talk” between adjacent or even distant microwells.

Consider the case of a two-dimensional, planar array of reaction sites in contact with a bulk fluid (e.g., a solution containing a common reagent or a wash solvent), and presume that at least one of the reactants and/or products involved in the chemistry at a particular reaction site is soluble in the bulk fluid. (Alternatively, consider the case of an array of microvessels that all open out onto a common fluid; the analyses are similar.) In the absence of convective flow of bulk fluid, transport of reaction participants (and cross-contamination or “cross-talk” between adjacent reaction sites or microvessels) can take place only by diffusion. If the reaction site is considered to be a point source on a 2-D surface, the chemical species of interest (e.g., a reaction product) will diffuse radially from the site of its production, creating a substantially hemispherical concentration field above the surface (see FIG. 1).

The distance that a chemical entity can diffuse in any given time t may be estimated in a crude manner by considering the mathematics of diffusion (Crank, 1975). The rate of diffusive transport in any given direction x (cm) is given by Fick's law as

$$j = - D \frac{\partial C}{\partial x} \qquad Eq . 1$$

where j is the flux per unit area (g-mol/cm²-s) of a species with diffusion coefficient D (cm²/s), and ∂C/∂x is the concentration gradient of that species. The mathematics of diffusion are such that a characteristic or “average” distance an entity can travel by diffusion alone scales with the one-half power of both the diffusion coefficient and the time allowed for diffusion to occur. Indeed, to order of magnitude, this characteristic diffusion distance can be estimated as the square root of the product of the diffusion coefficient and time—as adjusted by a numerical factor of order unity that takes into account the particulars of the system geometry and initial and/or boundary conditions imposed on the diffusion process.

It will be convenient to estimate this characteristic diffusion distance as the root-mean-square distance dᵣₘₛ that a diffusing entity can travel in time t:
dᵣₘₛ=\sqrt{2Dt} Eq. 2
As stated above, the distance that a diffusing chemical typically travels varies with the square root of the time available for it to diffuse—and inversely, the time required for a diffusing chemical to travel a given distance scales with the square of the distance to be traversed by diffusion. Thus, for a simple, low-molecular-weight biomolecule characterized by a diffusion coefficient D of order 1·10⁻⁵ cm²/s, the root-mean-square diffusion distances dᵣₘₛ that can be traversed in time intervals of 0.1 s, 1.0 s, 2.0 s, and 10 s are estimated by means of Equation 2 as 14 μm, 45 μm, 63 μm, and 141 μm, respectively.

Such considerations place an upper limit on the surface density or number per unit area of microwells or reaction sites that can be placed on a 2-D surface if diffusion of chemicals from one microwell or reaction site to an adjacent well or site (and thus cross-contamination) is to be minimized. More particularly, given that the species diffusivity and the time available for diffusion are such that dᵣₘₛ is the characteristic diffusion distance as estimated with Equation 2, it is evident that adjacent microwells or reaction sites can be spaced no more closely to one another than a fraction of this distance dᵣₘₛ if diffusion of reaction participants between them is to be held to a minimum. This, then, restricts the numbers of reaction sites that can be placed on a 2-D surface. More precise calculations of the actual concentration of a diffusing species at an adjacent microwell or reaction site can be performed by solving—with either analytical or numerical methods—the partial differential equations that describe unsteady-state diffusion subject to appropriate initial and boundary conditions (Crank, 1975). However, the order of magnitude analysis provided here suffices to illustrate the magnitude of the problem that must be solved if multiple, parallel reactions are to be conducted independently in a high-density format without risk of chemical cross-talk or contamination from nearby reaction centers.

The issue of contamination of a reaction center or well by chemical products being generated at nearby reaction centers or microwells becomes even more problematic when reaction sites are arrayed on a 2-D surface (or wells are arranged in an essentially two-dimensional microtiter plate) over which a fluid flows (again, see FIG. 1). In this case, compounds produced at a surface reaction site or within a well undergo diffusive transport up and away from the surface (or out of the reaction wells), where they are subsequently swept downstream by convective transport of fluid that is passing through a flow channel in fluid communication with the top surface of the array.

Several options exist for decreasing the spacing between (and thus increasing the number per unit area) of reaction sites. For example:

(1) Discrete reaction centers can be connected with microscopic tubes or channels in a “microfluidics” approach as described, e.g., by Cherukuri et al. (1999). However, this approach entails complex microfabrication, assembly of microcomponents, and control of fluid flow.

(2) The reaction centers can be placed at the bottom of microwells, such that dᵣₘₛ is arranged to be small as compared to the sum of the depth of the microwell plus the spacing between adjacent microwells. Such microwells can be formed, for example, by microfabrication or microprinting (e.g. Aoki, 1992; Kane et al., 1999; Dannoux et al., 2000; Deng et al., 2000; Zhu et al., 2000), or by etching the ends of a fused fiber optic bundle (e.g. Taylor and Walt, 2000; Illumina Inc.; 454 Corporation—see, e.g., U.S. Pat. No. 6,274,320, incorporated fully herein by reference). In these etched wells, the distance from the top to the bottom of the microwells must be traversed not only by reaction products (the escape of which it is desired to minimize) but by reactants as well (whose access it is desired not to impede). That is, if a reaction is confined to the base of a microwell, reactants must traverse the distance from the top to the bottom of the microwells by diffusion, potentially reducing the rate of reactant supply and possibly limiting the rate of reaction.

(3) The space between reaction centers can be filled with a medium in which the diffusing chemical has low diffusivity, thus reducing the rate of transport of said compound to adjacent centers. Again, however, this adds complexity and may impede (i.e., slow) access of reactant to the reaction site.

(4) If the diffusing species is charged, it may be possible to establish an electric field so as to counter diffusion, as exemplified, e.g., by Nanogen, Inc. Creation of the appropriate electrodes, however, again adds to the complexity of fabrication, and regulation of voltages at the electrodes adds complexity to the control system.

### SUMMARY OF THE INVENTION

An alternative technique for densely packing microreactors in a substantially 2-D arrangement is described here. This technique provides not only dense, two-dimensional packing of reaction sites, microvessels, and reaction wells—but also provides for efficient delivery of reagents and removal of products by convective flow rather than by diffusion alone. This latter feature permits much more rapid delivery of reagents and other reaction auxiliaries—and it permits faster and more complete removal of reaction products and by-products—than has heretofore been possible using methods and apparatus described in the prior art.

In one aspect, the invention includes a confined membrane reactor array (CMRA) comprising (a) a microreactor element comprising an array of open microchannels or open microwells, the longitudinal axes of said microchannels or microwells arranged in a substantially parallel manner; and (b) a porous filter element in contact with the microreactor element to form a bottom to the microchannels or microwells, thereby defining a series of reaction chambers, wherein the porous filter element comprises a permselective membrane that blocks the passage of nucleic acids, proteins and beads there across, but permits the passage of low molecular weight solutes, organic solvents and water there across. In a preferred embodiment, the microreactor element comprises a plate formed from a fused fiber optic bundle, wherein the microchannels extend from the top face of the plate through to the bottom face of the plate. In another embodiment, the CMRA further comprises an additional porous support between the microreactor element and the porous filter element. In one embodiment, the porous filter element comprises an ultrafilter. In a further embodiment, the CMRA further comprises at least one mobile solid support disposed in each of a plurality of the microchannels of the microreactor element. The mobile solid support can be a bead. In a preferred embodiment, the mobile solid support has an enzyme and/or a nucleic acid immobilized thereon. In another aspect, the invention provides a method of making the CMRA comprising attaching a microreactor element to a porous filter element.

In a further aspect, the invention includes an unconfined membrane reactor array (UMRA) comprising a porous filter element against which molecules are concentrated by concentration polarization wherein discrete reaction chambers are formed in discrete locations on the surface of or within the porous filter element by depositing reactant molecules at discrete sites on or within the porous filter element. In a preferred embodiment, the reaction chambers are formed by depositing mobile solid supports having said reactant molecules immobilized thereon, on the surface of, or within, the porous element. In one embodiment, the porous filter element comprises an ultrafilter. In another embodiment, the mobile solid support is a bead. In another embodiment, the mobile solid support has an enzyme and/or a nucleic acid immobilized thereon.

In another aspect, the invention includes a UMRA comprising (a) a porous membrane with discrete reaction sites formed by depositing mobile solid supports having said reactant molecules thereon, on the surface of, or within the porous membrane; (b) a nucleic acid template immobilized to a solid support; and (c) optionally, at least one immobilized enzyme. As used herein, the term discrete reaction sites refers to individual reaction centers for localized reactions whereby each site has unique reactants and products such that there is no cross-contamination between adjacent sites. In one embodiment, the mobile solid support is a bead. In another embodiment, the porous membrane is a nylon membrane. In another embodiment, the porous membrane is made of a woven fiber. In a preferred embodiment, the porous membrane pore size at least 0.02 μm. In another embodiment, the solid support is selected from the group consisting of a bead, glass surface, fiber optic or the porous membrane. In another embodiment, the immobilized enzyme is immobilized to a bead or the porous membrane. In one embodiment, the immobilized enzyme is selected from the group consisting of ATP sulfurylase luciferase, hypoxanthine phosphoribosyltransferase, xanthine oxidase, uricase or peroxidase.

In another aspect, the invention includes an array comprising (a) a first porous membrane with a plurality of discrete reaction sites disposed thereon, and/or within, wherein each reaction site has immobilized template adhered to the surface; and (b) a second porous membrane with at least one enzyme located on the surface of, and/or within, the membrane, wherein the second porous membrane is in direct contact with the first porous membrane.

In another aspect, the invention provides a CMRA comprising an array of open microchannels or microwells attached to a porous filter or membrane. In one embodiment, the CMRA further comprises a mechanical support, wherein the mechanical support separates the microchannels from the porous membrane. In a preferred embodiment, the mechanical support is selected from the group consisting of plastic mesh, wire screening or molded or machined spacers.

In another aspect, the invention includes an apparatus for determining the nucleic acid sequence in a template nucleic acid polymer, comprising: (a) a CMRA or UMRA; (b) nucleic acid delivery means for introducing template nucleic acid polymers to the discrete reaction sites; (c) nucleic acid delivery means to deliver reagents to the reaction sites to create a polymerization environment in which the nucleic acid polymers will act as template polymers for the synthesis of complementary nucleic acid polymers when nucleotides are added; (d) convective flow delivery means to immobilize reagents to the porous membrane; (e) detection means for detecting the formation of inorganic pyrophosphate enzymatically; and (f) data processing means to determine the identity of each nucleotide in the complementary polymers and thus the sequence of the template polymers. In one embodiment, the detection means is a CCD camera. In another embodiment, the data processing means is a computer.

In another aspect, the invention provides an apparatus for processing a plurality of analytes, the apparatus comprising: (a) a CMRA or an UMRA; (b) fluid means for delivering processing reagents from one or more reservoirs to the flow chamber so that the analytes disposed therein are exposed to the reagents; and (c) detection means for detecting a sequence of optical signals from each of the reaction sites, each optical signal of the sequence being indicative of an interaction between a processing reagent and the analyte disposed in the reaction site, wherein the detection means is in communication with the reaction site. In one embodiment, the convective flow delivery means is a peristaltic pump.

In another aspect, the invention includes an apparatus for determining the base sequence of a plurality of nucleotides on an array, the apparatus comprising: (a) a CMRA or UMRA; (b) reagent delivery means for adding an activated nucleotide 5′-triphosphate precursor of one known nitrogenous base to a reaction mixture to each reaction site, each reaction mixture comprising a template-directed nucleotide polymerase and a single-stranded polynucleotide template hybridized to a complementary oligonucleotide primer strand at least one nucleotide residue shorter than the templates to form at least one unpaired nucleotide residue in each template at the 3′-end of the primer strand, under reaction conditions which allow incorporation of the activated nucleoside 5′-triphosphate precursor onto the 3′-end of the primer strands, provided the nitrogenous base of the activated nucleoside 5′-triphosphate precursor is complementary to the nitrogenous base of the unpaired nucleotide residue of the templates; (c) detection means for detecting whether or not the nucleoside 5′-triphosphate precursor was incorporated into the primer strands in which incorporation of the nucleoside 5′-triphosphate precursor indicates that the unpaired nucleotide residue of the template has a nitrogenous base composition that is complementary to that of the incorporated nucleoside 5′-triphosphate precursor; (d) means for sequentially repeating steps (b) and (c), wherein each sequential repetition adds and detects the incorporation of one type of activated nucleoside 5′-triphosphate precursor of known nitrogenous base composition; and (e) data processing means for determining the base sequence of the unpaired nucleotide residues of the template in each reaction chamber from the sequence of incorporation of said nucleoside precursors.

In another aspect, the invention includes an apparatus for determining the nucleic acid sequence in a template nucleic acid polymer, comprising: (a) a CMRA or UMRA; (b) nucleic acid delivery means for introducing a template nucleic acid polymers onto the reaction sites; (c) nucleic acid delivery means to deliver reagents to the reaction chambers to create polymerization environment in which the nucleic acid polymers will act as a template polymers for the synthesis of complementary nucleic acid polymers when nucleotides are added; (d) reagent delivery means for successively providing to the polymerization environment a series of feedstocks, each feedstock comprising a nucleotide selected from among the nucleotides from which the complementary nucleic acid polymer will be formed, such that if the nucleotide in the feedstock is complementary to the next nucleotide in the template polymer to be sequenced said nucleotide will be incorporated into the complementary polymer and inorganic pyrophosphate will be released; (e) detection means for detecting the formation of inorganic pyrophosphate enzymatically; and (f) data processing means to determine the identity of each nucleotide in the complementary polymers and thus the sequence of the template polymers.

In one aspect, the invention includes a system for sequencing a nucleic acid comprising the following components: (a) a CMRA or UMRA; (b) at least one enzyme immobilized on a solid support; (c) means for flowing reagents over said porous membrane; (d) means for detection; and (e) means for determining the sequence of the nucleic acid.

In a further aspect, the invention includes a system for sequencing a nucleic acid comprising the following components: (a) a CMRA or UMRA; (b) at least one enzyme immobilized on a solid support; (c) means for flowing reagents over said porous membrane; (d) means for detection; and (e) means for determining the sequence of the nucleic acid.

In another aspect, the invention provides a method for carrying out separate parallel independent reactions in an aqueous environment, comprising: (a) delivering a fluid containing at least one reagent to an array, using the CMRA of claim 1 or the UMRA of claim 9, wherein each of the reaction sites immersed in a substance such that when the fluid is delivered onto each reaction site, the fluid does not diffuse onto an adjacent site; (b) washing the fluid from the array in the time period after the starting material has reacted with the reagent to form a product in each reaction site; (c) sequentially repeating steps (a) and (b). In one embodiment, the product formed in any one reaction chamber is independent of the product formed in any other reaction chamber, but is generated using one or more common reagents. In another embodiment, the starting material is a nucleic acid sequence and at least one reagent in the fluid is a nucleotide or nucleotide analog. In a preferred embodiment, the fluid additionally comprises a polymerase capable of reacting the nucleic acid sequence and the nucleotide or nucleotide analog. In another embodiment, the method additionally comprises repeating steps (a) and (b) sequentially. In one embodiment, the substance is mineral oil. In a further embodiment, the reaction sites are defined by concentration polarization.

In one aspect, the invention includes a method of determining the base sequence of nucleotides in an array format, the method comprising the steps of: (a) adding an activated nucleoside 5′-triphopsphate precursor of one known nitrogenous base composition to a plurality of reaction sites localized on a CMRA or UMRA, wherein the reaction site is comprised of a template-directed nucleotide polymerase and a heterogenous population of single stranded templates hybridized to complementary oligonucleotide primer strands at least one nucleotide residue shorter than the templates to form at least one unpaired nucleotide residue in each template at the 3′ end of the primer strand under reaction conditions which allow incorporation of the activated nucleoside 5′-triphosphate precursor onto the 3′ end of the primer strand under reaction conditions which allow incorporation of the activated nucleoside 5′-triphosphate precursor onto the 3′ end of the primer strands, provided the nitrogenous base of the activated nucleoside 5′-triphosphate precursor is complementary to the nitrogenous base of the unpaired nucleotide residue of the templates; (b) detecting whether or not the nucleoside 5′-triphosphate precursor was incorporated into the primer strands in which incorporation of the nucleoside 5′-triphosphate precursor indicates that the unpaired nucleotide residue of the template has a nitrogenous base composition that is complementary to that of the incorporated nucleoside 5′-triphosphate precursor; and (c) sequentially repeating steps (a) and (b), wherein each sequential repetition adds and detects the incorporation of one type of activated nucleoside 5′-triphosphate precursor of known nitrogenous base composition; (d) determining the base sequence of the unpaired nucleotide residues of the template from the sequence of incorporation of said nucleoside precursors.

In a preferred embodiment, the detection of the incorporation of the activated precursor is accomplished enzymatically. The enzyme utilized can be selected from the group consisting of ATP sulfurylase, luciferase, hypoxanthine phosphoribosyltransferase, xanthine oxidase, uricase or peroxidase. In one embodiment, the enzyme is immobilized to a solid support. In another embodiment, the solid support is selected from the group comprising a bead, glass surface, fiber optic or porous membrane.

In a further aspect, the invention includes a method of determining the base sequence of a plurality of nucleotides on an array, said method comprising: (a) providing a plurality of sample DNA's, each disposed within a plurality of reaction sites on a CMRA or UMRA; (b) detecting the light level emitted from a plurality of reaction sites on respective proportional of an optically sensitive device; (c) converting the light impinging upon each of said portions of said optically sensitive device into an electrical signal which is distinguishable from the signals from all of said other regions; (d) determining a light intensity for each of said discrete regions from the corresponding electrical signal; (e) recording the variations of said electrical signals with time.

### BRIEF DESCRIPTION OF THE DRAWINGS

FIG. 1: Effect of flow across a 2-D array of microwells. On the left, there is no flow, and diffusion of compound (grey) creates a hemispherical chemical concentration gradient emanating from the microwell containing the reaction. On the right, flow carries compound downstream, creating a concentration plume. Cross-contamination of microwells resulting from diffusive and/or convective transport of compound to nearby and/or distant wells is minimized or avoided by the present invention.

FIG. 2: An integral or physical composite of a microchannel array and a porous membrane barrier forming a confined membrane reactor array (CMRA). The flow of fluid through the CMRA carries reaction participants along with it. Rejected macromolecules experience concentration polarization and so are concentrated and localized within the microchannels, without their being immobilized on a support.

FIG. 3: Sequential addition of solutions containing different macromolecules permits their stratification within microchannels or microwells, thus forming the microscopic equivalent of a stacked column.

FIG. 4: Fluid flow atop and tangential to the CMRA surface may cause a pressure drop along the flow compartment that, in turn, may cause the pressure difference across the CMRA to decrease with distance along it. If this variation is significant, the flow rate through the CMRA will also vary with distance from the inlet, causing the delivery of molecules to the microchannels to be nonuniform.

FIG. 5: Radial or circumferential fluid inlets reduce the pressure variation over the CMRA, more nearly equalizing flow rates through the microchannels.

FIG. 6: Fluid flow restrictor, valve, or back-pressure regulator downstream of CMRA provides dominant flow resistance (i.e., large pressure drop) as compared to that associated with CMRA flow compartment—thus maintaining relatively uniform pressure difference across (and uniform fluid flow through) the CMRA. Shown here with optional fluid recirculation.

FIG. 7: An unconfined membrane reactor array (UMRA). Certain molecules may be concentrated adjacent to the porous filter by concentration polarization. Other molecules or particles are added in such a manner as to form a dispersed 2-D array of discrete reaction sites on or within the UMRA. Products formed at discrete reaction sites are carried by flow through the porous filter (e.g., a UF membrane), creating a plume of reaction products that extends downstream. Products are swept out of the UMRA before separate plumes merge, thus effectively minimizing or avoiding cross-contamination of independent reactions meant to occur at different reaction sites.

FIG. 8: An unconfined membrane reactor array (UMRA) comprised of an ultrafiltration membrane and a coarse filter, mesh, or other grossly porous matrix. The two filters are sandwiched together, with the coarser filter upstream. The latter can assist in stabilizing the layer of concentration-polarized molecules formed adjacent to the ultrafiltration membrane, and it can provide some resistance to lateral diffusion of reaction products. The coarser filter, mesh, or matrix may also provide mechanical support.

FIG. 9: A schematic of a pyrophosphate-based sequencing method with photon detection.

FIG. 10: Use of a CCD as a photodetector array to detect light production from a microchannel or microwell in a CMRA.

FIG. 11: Experimental setup for the convective flow embodiment described in Example 1.

FIG. 12: Effect of immobilization of the luciferase and ATP sulfurylase on the sepharose beads with Oligo seq 1. (A) Enzymes have not been immobilized on the beads. (B) Enzymes have been immobilized on the beads and the signal has been improved by factor of 3.5 times.

FIG. 13: An scanning electron micrograph (SEM) photo of the nylon weave filter that can be utilized for the CMRA and UMRA.

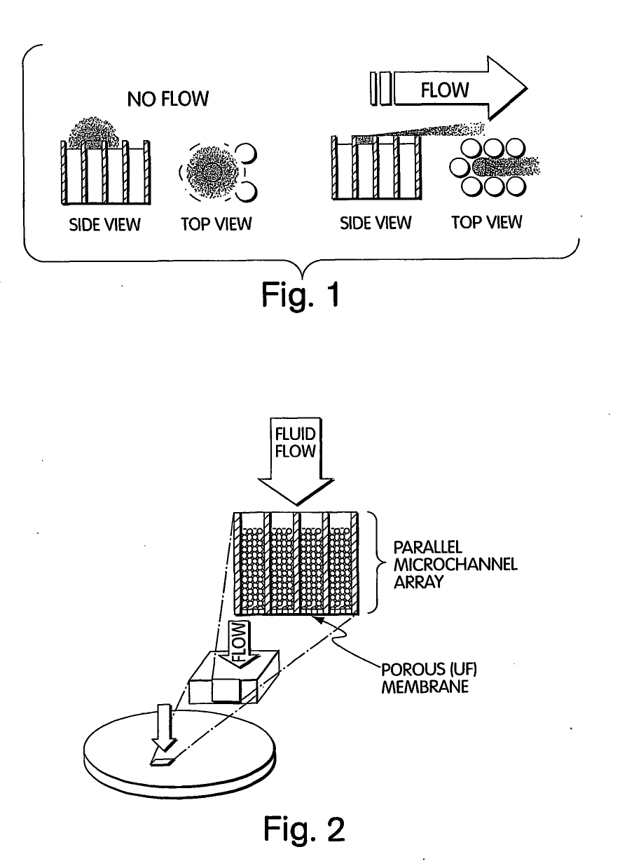

### DETAILED DESCRIPTION OF THE INVENTION

Methods and apparati are described here for providing a dense array of discrete reaction sites, microreactor vessels, and/or microwells in a substantially two-dimensional configuration (see FIG. 2) and for charging such microreactors with reaction participants by effecting a convective flow of fluid normal to the plane of and through the array of reaction sites or microvessels. Reaction participants that may be charged to, concentrated, and contained within said reaction sites or microreactor vessels by methods of the present invention include high-molecular-weight reactants, catalysts, and other reagents and reaction auxiliaries. In the context of oligonucleotide sequencing and DNA/RNA analysis, such high-molecular-weight reactants include, for example, oligonucleotides, longer DNA/RNA fragments, and constructs thereof. These reactants may be free and unattached (if their molecular weight is sufficient to permit them to be contained by the method of the present invention), or they may be covalently bound to or otherwise associated with, e.g., high-molecular-weight polymers, high-surface-area beads or gels, or other supports known in the art. Examples of reaction catalysts that may be similarly delivered to and localized within said reaction sites or microvessels include enzymes, which may or may not be associated with or bound to solid-phase supports such as porous or non-porous beads.

The present invention also includes means for efficiently supplying relatively lower-molecular-weight reagents and reactants to said discrete reaction sites or microreactor vessels—as well as means for efficiently removing unconverted reactants and reaction products from said reaction sites or microvessels. More particularly, efficient reagent delivery and product removal are accomplished in the present invention by arranging for at least some convective flow of solution to take place in a direction normal to the plane of the substantially two-dimensional array of reaction sites or microreactor vessels—and thus past or through the discrete sites or microvessels, respectively, where chemical reaction takes place. In this instance, reactants and products will not necessarily be retained or concentrated at the reaction sites or within the reaction microvessels or microwells; indeed, it may be desired that certain reaction products be rapidly swept away from and/or out of said reaction sites or microvessels.

In addition to including means for providing a controlled convective flux of fluid normal to and across the substantially planar array of reaction sites or microreactors, the present invention also includes permselective, porous filter means capable of discriminating between large (i.e., high-molecular-weight) and small (i.e., low-molecular-weight) reaction participants. This filter means is capable of selectively capturing or retaining certain reaction participants while permitting others to be flushed through and/or out the bottom of the microreactor array. By the proper selection of porous filter and the judicious choice of convective flux rates, considerable control over the location, concentration, and fate of reaction participants can be realized.

CMRAs.

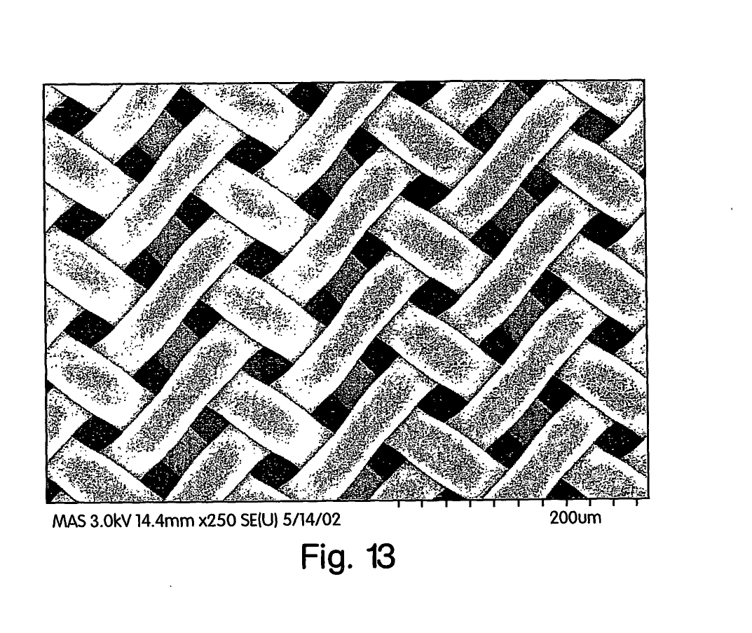

In a preferred embodiment, the apparatus of the present invention consists of an array of microreactor elements comprised of at least two functional elements that may take various physical or structural forms—namely, (i) a microreactor element comprised of an array of microchannels or microwells, and (ii) a porous filter element comprising, e.g., a porous film or membrane in the form of a sheet or thin layer (see, e.g., FIG. 13). These two elements are arranged next to and in close proximity or contact with one another, with the plane of the microchannel/microvessel element parallel to the plane of the porous filter element. For the sake of definiteness, the side of this composite structure containing the microchannel or microvessel array will be referred to hereinafter as the “top”, while the side defined by the porous filter will be referred to as the “bottom” of the structure.

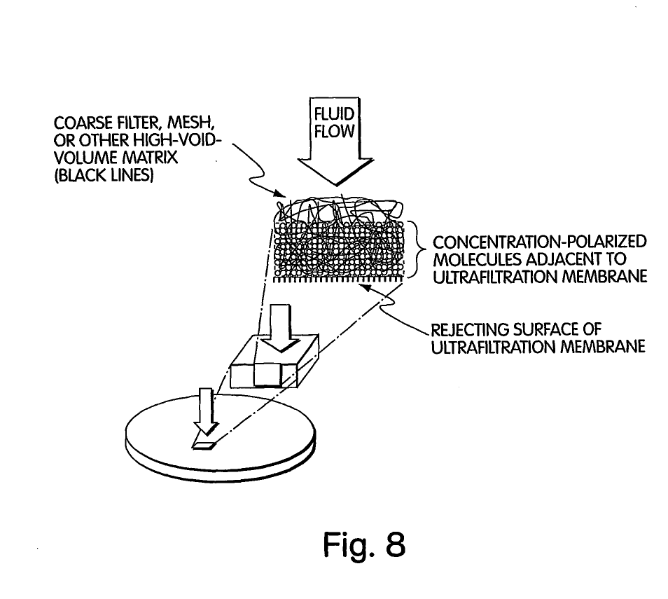

The microchannel or microvessel element consists of a collection of numerous microchannels, with the longitudinal axes of said microchannels being arranged in a substantially parallel manner, and with the downstream ends of said channels being in functional contact with a porous membrane or other filter element. The porous filter or membrane is chosen to be permselective—i.e., to block the passage of certain species like particles, beads, or macromolecules (e.g., proteins and DNA), while permitting the passage of relatively low-molecular-weight solutes, organic solvents, and water. The aspect ratio of the microchannels (i.e., their height- or length-to-diameter ratio) may be small or large, and their cross-section may take any of a number of shapes (e.g., circular, rectangular, hexagonal, etc.). As discussed further below, it is not at all essential that the microchannel walls be continuous or regular; indeed, highly porous, “spongy” matrices with interconnecting pores communicating between adjacent channels will also serve functionally as “microchannel” elements, despite the fact that they will not necessarily contain discrete or functionally confined or isolated microchannels per se. (This embodiment is described in more detail in discussion that follows and in FIG. 8).

In many cases it will be appropriate to consider the entire array assembly (i.e., the combination of microchannel/microvessel element plus porous filter element) as a single substantially two-dimensional structure comprised of either an integral or a physical composite, as described further below. For the sake of brevity, all such systems of the present invention that are comprised of composite structures containing at minimum a microchannel or microvessel element and a porous filter or membrane element will be referred to henceforth as “confining membrane reactor arrays” or CMRAs. The CMRAs of the present invention will be seen to possess some of the general structural features and functional attributes of commercially available microtiter filter plates of the sort commonly used in biology laboratories, wherein porous filter disks are molded or otherwise incorporated into the bottoms of plastic wells in 96-well plates. However, the CMRA is differentiated from these by the unparalleled high density of discrete reaction sites that it provides, by its unique construction, and by the novel and uniquely powerful way in which it can be operated to perform high-throughput chemistries—for example, of the sorts applied to the amplification and/or analysis of DNA.

The composite microreactor/filter structure—i.e., the CMRA of the present invention—can take several physical forms; as alluded to above, two such forms are represented by physical composites and integral composites, respectively. As regards the former, the two functional elements of the structure—that is, the microchannel array and the porous filter—are provided as separate parts or components that are merely laid side-by-side, pressed together, or otherwise attached in the manner of a sandwich or laminate. This structural embodiment will be referred to hereinafter as a “physical composite”. Additional porous supports (e.g., fine wire mesh or very coarse filters) and/or spacing layers may also be provided where warranted to provide mechanical support for the finely porous filter element and to ensure good contact between the microchannel and porous filter elements. Plastic mesh, wire screening, molded or machined spacers, or similar structures may be provided atop the CMRA to help define a compartment for tangential flow of fluid across the top of the CMRA, and similar structures may be provided beneath the CMRA to provide a pathway for egress of fluid that has permeated across the CMRA.

Alternatively, the two functional elements of the CMRA may be part and parcel of a single, one-piece composite structure—more particularly, an “integral composite”. An integral composite has one surface that is comprised of a finely porous “skin” region that is permselective—i.e., that permits solvent and low-molecular-weight solutes to permeate, but that retains or rejects high-molecular-weight solutes (e.g., proteins, DNA, etc.), colloids, and particles. However, the bulk of this structure's through-thickness will be comprised of microchannels and/or large voids or macropores that are incapable of exhibiting permselectivity by virtue of the very large size of the microchannels or voids contained therein.

Many synthetic membranes of the type employed in ultrafiltration processes and generally known as “ultrafilters” are known in the art and can be described as “integral composites” for present purposes (Kulkarni et al., 1992; Eykamp, 1995). Ultrafiltration membranes are generally regarded as having effective pore sizes in the range of a nanometer or so up to at most a hundred nanometers. As a consequence, ultrafilters are capable of retaining species with molecular weights ranging from several hundred Daltons to several hundred thousand Daltons and up. Most UF membranes are described in terms of a nominal molecular-weight cut-off (MWCO). The MWCO can be defined in various ways, but commonly a membrane's MWCO corresponds to the molecular weight of the smallest species for which the membrane exhibits greater than 90% rejection.

Many ultrafilters are asymmetric. That is, they are characterized by having a thin (micron- or even submicron-thick) skin layer containing nanometer-size pores—said skin layer being integrally supported by and inseparable from a much thicker substrate region of order 100 μms and more in thickness, and with said substrate region having pore diameters that are generally at least an order of magnitude larger than those in the skin. Whereas the pores in the skin region of an ultrafiltration membrane that give rise to its permselectivity are typically in the range from a few to a few hundred nanometers, the voids in the substrate region of said ultrafilters might be as large as a few tenths of a micron to many microns in diameter. Most polymeric ultrafiltration membranes are generally prepared in a single membrane casting step, with both the ultraporous skin layer and the substrate region necessarily comprised of the same, continuous material.

An inorganic membrane filter with utility as a confined membrane reactor array (CMRA) of the integral composite type is exemplified by the Anopore™ and Anodisc™ families of ultrafiltration membranes sold, for example, by Whatman PLC (see, for instance, http://www.whatman.plc.uk/index2.html). These high-purity alumina membranes are prepared by an electrochemical oxidation process that gives rise to a rather unique membrane morphology (Furneaux et al., 1989; Martin, 1994; Mardilovich et al., 1995; Asoh et al., 2001). In particular, such membranes provide both an array of parallel microchannels capable of housing independent reactions and a more finely porous permselective surface region capable of rejecting selected reaction participants. Commercially available alumina membranes (e.g., from Whatman) contain a densely packed array of regular, nearly hexagonal-shaped channels, nominally 0.2 μm in diameter, with no lateral crossovers between adjacent channels. The membranes have an overall thickness of about 60 μm, with almost the entire thickness being comprised of these 0.2-μm-diameter channels. However, on one surface is located a much more finely porous—even ultraporous—surface region of order 1 μm in thickness, said surface region containing pores characterized by pore diameters of about 0.02 μm or 20 nm—i.e., in the ultrafiltration range. These membranes have the additional interesting and useful optical property of being substantially transparent when wet with aqueous solutions—a feature that permits any light generated by chemical reaction within them to be readily detected.

In conventional applications where Anopore™ or Anodisc™ membranes are used to concentrate and/or separate proteins by ultrafiltration, the higher fluid pressure will normally be applied to the side of the membrane characterized by the smaller pores. That is, fluid generally is made to flow first through the thin, permselective surface region of the UF membrane and only then through the much thicker substrate region with its larger, substantially parallel microchannels; in this event, the substrate region of the membrane serves merely to provide physical support and mechanical integrity. As explained in more detail below, however, the use of membranes of this type in CMRAs entails reversing the direction of convective flow through them, such that fluid flows first through the thick substrate region containing the parallel microchannels within which reaction occurs—and only then through the more finely porous and permselective surface region. In a sense, then, these ultrafiltration membranes are oriented “upside-down” (i.e., rejecting side “down” and opposite the higher-pressure side) when used as CMRAs—at least as compared to their more usual orientation in ordinary ultrafiltration applications.

Alternatively, ultrafiltration membranes may also be used as CMRA components with their more finely porous, rejecting side “up”—i.e., in contact with the higher fluid pressure—such that fluid flows first through the permselective skin region and then through the more grossly porous, spongy substrate region. However, in such instances it will generally be the case that the CMRA will be of the “physical composite” type—with a separate and distinct microchannel- or microvessel-containing element placed “above” and in intimate contact with the skin region of the ultrafilter. In this case, the order of fluid flow will be first through the microchannel-containing element, then across the thin skin of the permselective region of the integral composite UF membrane, and then finally across the substrate region of the UF membrane. Additional supporting layers may optionally be provided underneath the physical composite-type CMRA, and plastic mesh, wire screens, or like materials may be used atop the composite to help define a compartment for tangential flow of fluid across the top of the CMRA as before.

In contrast to the operation of many prior-art microreactor arrays, wherein diffusion of reactants into and products back out of an array of microvessels occurs solely by diffusion, the operation of the confined membrane reactor arrays of the present invention entails providing for a modest convective flux through the CMRA. In particular, a small pressure difference is applied from the top to the bottom surface of the CMRA sufficient to establish a controlled convective flux of fluid through the structure in a direction normal to the substantially planar surface of the structure. Fluid is thus made to flow first through the microchannel element and then subsequently across the porous filter element. This convective flow enables the loading and entrapment within the microchannel element of the CMRA of various high-molecular-weight reagents and reaction auxiliaries that are retained by the porous filter element of the CMRA. By the same token, this convective flow enables the rapid delivery to the site of reaction of low-molecular-weight reactants—and the efficient and complete removal of low-molecular-weight reaction products from the site of their production: Particularly important is the fact that the convective flow serves to impede or substantially prevent the back-diffusion of reaction products out of the upstream ends of the microchannels, where otherwise they would be capable of contaminating adjacent or even distant microreactor vessels.

The relative importance of convection and diffusion in a transport process that involves both mechanisms occurring simultaneously can be gauged with the aid of a dimensionless number—namely, the Peclet number Pe. This Peclet number can be viewed as a ratio of two rates or velocities—namely, the rate of a convective flow divided by the rate of a diffusive “flow” or flux. More particularly, the Peclet number is a ratio of a characteristic flow velocity V(in cm/s) divided by a characteristic diffusion velocity D/L (also expressed in units of cm/s)—both taken in the same direction:

$$Pe = \frac{VL}{D} \qquad Eq . 3$$

In Equation 3, V is the average or characteristic speed of the convective flow, generally determined by dividing the volumetric flow rate Q (in cm³/s) by the cross-sectional area A (cm²) available for flow. The characteristic length L is a representative distance or system dimension measured in a direction parallel to the directions of flow and of diffusion (i.e., in the direction of the steepest concentration gradient) and selected to be representative of the typical or “average” distance over which diffusion occurs in the process. And finally D (cm²/s) is the diffusion coefficient for the diffusing species in question. (An alternative but equivalent formulation of the Peclet number Pe views it as the ratio of two characteristic times—namely, of representative times for diffusion and convection. Equation 3 for the Peclet number can equally well be obtained by dividing the characteristic diffusion time L²/D by the characteristic convection time L/V.)

The convective component of transport can be expected to dominate over the diffusive component in situations where the Peclet number Pe is large compared to unity. Conversely, the diffusive component of transport can be expected to dominate over the convective component in situations where the Peclet number Pe is small compared to unity. In extreme situations where the Peclet number is either very much larger or very much smaller than one, transport may be accurately presumed to occur either by convection or by diffusion alone, respectively. Finally, in situations where the estimated Peclet number is of order unity, then both convection and diffusion can be expected to play significant roles in the overall transport process.

The diffusion coefficient of a typical low-molecular-weight biomolecule will generally be of the order of 10⁻⁵ cm²/s (e.g., 0.52·10⁻⁵ cm/s for sucrose, and 1.06·10⁻⁵ cm/s for glycine). Thus, for chemical reaction sites, microchannels, or microvessels separated by a distance of 100 μm (i.e., 0.01 cm), the Peclet number Pe for low-molecular-weight solutes such as these will exceed unity for flow velocities greater than about 10 μm/sec (0.001 cm/s). For sites or vessels separated by only 10 μm (i.e., 0.001 cm), the Peclet number Pe for low-molecular-weight solutes will exceed unity for flow velocities greater than about 100 μm/sec (0.01 cm/s). Convective transport is thus seen to dominate over diffusive transport for all but very slow flow rates and/or very short diffusion distances.

Where the molecular weight of a diffusible species is substantially larger—for example as it is with large biomolecules like DNA/RNA, DNA fragments, oligonucleotides, proteins, and constructs of the former—then the species diffusivity will be corresponding smaller, and convection will play an even more important role relative to diffusion in a transport process involving both mechanisms. For instance, the aqueous-phase diffusion coefficients of proteins fall in about a 10-fold range (Tanford, 1961). Protein diffusivities are bracketed by values of 1.19·10⁻⁶ cm²/s for ribonuclease (a small protein with a molecular weight of 13,683 Daltons) and 1.16·10⁻⁷ cm²/s for myosin (a large protein with a molecular weight of 493,000 Daltons). Still larger entities (e.g., tobacco mosaic virus or TMV at 40.6 million Daltons) are characterized by still lower diffusivities (in particular, 4.6·10⁻⁸ cm²/s for TMV) (Lehninger, 1975). The fluid velocity at which convection and diffusion contribute roughly equally to transport (i.e., Pe of order unity) scales in direct proportion to species diffusivity.

With the aid of the Peclet number formalism it is possible to gauge the impact of convection on reactant supply to—and product removal from—microreactor vessels. On the one hand, it is clear that even modest convective flows can appreciably increase the speed at which reactants are delivered to the interior of the microchannels or microwells in a CMRA structure. In particular, suppose for the sake of simplicity that the criteria for roughly equal convective and diffusive flows is considered to be Pe=1. One may then estimate that a convective flow velocity of the order of only 0.004 cm/s will suffice to carry reactant into a 25-μm-deep well at roughly the same rate as it could be supplied to the bottom of the well by diffusion alone, given an assumed value for reactant diffusivity of 1·10⁻⁵ cm²/s. The corresponding flow velocity required to match the rate of diffusion of such a species from the bottom to the top of a 2.5-μm-deep microwell is estimated to be of order 0.04 cm/s. Clearly, flow velocities through the CMRA much higher than this are possible, thereby illustrating the degree to which a modest convective flow can augment the diffusive supply of reactants to CMRA microchannels and wells.

By the same token, the Peclet number formalism assists in understanding how effective even a modest trans-CMRA convective flow component can be in impeding or substantially preventing the back-diffusion of excess unconverted reactants and/or reaction products and by-products into the flow compartment located above the “top” or upstream surface of the CMRA. Preventing such back-diffusion is critical since, once a compound escapes into this transverse flow compartment, it may readily diffuse and/or be swept along into the neighborhood of the mouths of adjacent microchannels, thus contributing to cross-contamination or cross-talk between reaction sites or microvessels. The magnitude of the “top-to-bottom” convective flow through the microvessels or microchannels of a CMRA that can be expected to have a significant effect in reducing the rate of diffusive compound loss out the top of the microchannels or microvessels can again be estimated to order of magnitude by setting the Peclet number equal to unity in Equation 3—with the understanding that, in this instance, the convective and diffusive flows will occur in opposite directions and oppose each other. The CMRA may be operated at microchannel Peclet numbers significantly greater than unity in situations where it is particularly critical that there be little or no escape of potential contaminant compounds from the top surface.

It may be noted that compounds which permeate across the porous filter element and out of the CMRA are made to flow straight-away out of the device, ideally in a direction substantially normal to the plane of the CMRA. By this and other fluid management strategies (e.g., the provision of thick, spongy pads underlying the CMRA), any potential for cross-contamination between nearby CMRA microchannels via the bottom surface of the structure may readily be avoided.

Thus far, it has been assumed that the freely diffusible reactants and products discussed in the paragraphs immediately above will experience only insignificant retention or rejection by the porous filter or membrane of the CMRA. The discussion turns now to focus on the fate of high-molecular-weight reaction participants and auxiliaries within the CMRA—in particular, of macromolecules including proteins, of oligo- and polynucleotides (and constructs thereof), and of otherwise low-molecular-weight reagents attached to high-molecular-weight polymers, nano- and micro-particles, or even beads. In these latter instances, said attachment facilitates the retention of reagents within the microvessels or microchannels of the CMRA.

A particularly useful feature of the present invention is its ability to permit the efficient and controlled loading of macromolecules and microparticles into said microvessels or microchannels by simple pressure-driven filtration across a suitably porous filter element. As discussed above, where the filter element has the ability to substantially reject and contain soluble macromolecules while permitting microsolutes to pass relatively freely, the filter is frequently referred to as an ultrafilter or ultrafiltration membrane—and the process is referred to as ultrafiltration.

Ultrafiltration (UF) is a process normally used to separate macromolecules from solutions according to the size and shape of the macromolecules relative to the pore size and morphology of the membrane. UF is a pressure-driven membrane permeation process, with flux of solvent (e.g., water) generally being proportional to an effective pressure difference that is equal to the applied hydraulic pressure difference ΔP (e.g., in atm) less any opposing osmotic pressure difference Δπ (in atm) that exists across the membrane by virtue of different solute concentrations in the feed or retentate relative to the permeate. The volumetric flux Jᵥ across the membrane (expressed in units of cm/s) is the volume of permeated fluid per unit of time and membrane area; it is given by the expression
Jᵥ=P·(ΔP−Δπ)/δ Eq. 4
where P is the membrane permeation coefficient or permeability (in cM²/s-atm)and δ (cm) is the effective thickness of the membrane.

UF membranes have nominal pore sizes ranging from about 1 nm on the low end to about 0.02 μm to at most 0.1 μm (i.e., 20 to 100 nm) on the high end, so solutes with molecular weights of several hundred or less can readily flow convectively through the pores under an applied pressure differences; species larger than the nominal molecular-weight cut-off (MWCO) are rejected and retained to a greater or lesser extent. The extent to which a given UF membrane is effective in retaining a particular solute species “i” can be expressed in terms of a rejection coefficient Rᵢ defined as
Rᵢ=1−(Cₚ/C_{b}) Eq. 5
where Cₚ is the solute concentration in the permeate and C_{b} is the solute concentration in the bulk solution (i.e., the feed or retentate in a separation application). (Equation 5 applies, strictly speaking, only in instances where boundary layer resistances are negligible and concentration polarization is insignificant. These considerations are discussed further below.) Low-molecular-weight solutes exhibit rejection coefficients close to zero, while macromolecular solutes with molecular weights well above the MWCO exhibit rejection coefficients approaching one. These concepts, normally applied to the ultrafiltrative separation and concentration of macromolecular solutes, are equally applicable to description of the structure and function of the porous membrane element in the contained membrane reactor arrays of the present invention.

When pressure-driven flux occurs through an ultrafiltration membrane; rejected macrosolutes accumulate at the high-pressure interface—normally at the high-pressure side of the rejecting skin layer of the UF membrane. As solutes accumulate, they are concentrated—not only within the bulk fluid but also within the thin fluid boundary later that normally resides at the high-pressure-side of the ultrafilter. The latter phenomenon is termed concentration polarization, and it is usually troublesome in conventional separation applications because it can reduce transmembrane flux (Lonsdale, 1982; Mulder, 1995; Cussler, 1997). However, in the CMRA applications of interest here, where the porous ultrafiltration element resides beneath a microchannel/microvessel element, concentration polarization can be a desirable phenomenon inasmuch as it provides a means for concentrating and effectively immobilizing high-molecular-weight reagents within the microchannels or microvessels of the CMRA. In effect, the microchannels or microvessels of a CMRA can be considered equivalent, structurally and functionally, to the stagnant film or fluid boundary layer that more typically resides atop the rejecting surface of an ultrafiltration membrane used to effect a separation.

The degree to which solutes are concentrated at the high-pressure interface (i.e., atop the permselective barrier that constitutes the skin region of an ultrafiltration membrane or functionally similar structure) can be estimated mathematically by considering the transport phenomena that give rise to concentration polarization. In ultrafiltration, solutes are continuously being carried to the surface of the membrane by the convective flow normal to the plane of the membrane, with solvent (typically, water) readily permeating the membrane. Those low-molecular-weight solutes that are not appreciably rejected (i.e., that have small Rᵢ values) readily permeate the membrane as well and experience little concentration at the interface. However, those solutes that are highly rejected (i.e., with Rᵢ values approaching unity) are blocked by the membrane and tend to diffuse away from its surface, back toward the main body or bulk of the fluid. Eventually a steady-state condition is reached, at which point the flow of solute towards the surface by convection is precisely balanced by the back-diffusion of solute to the bulk (and by solute leakage through the membrane to the extent that rejection is incomplete and Rᵢ<1). This balance is only established after the solute concentration gradient above the membrane (i.e., the driving force for diffusion) has become sufficiently steep. By writing the differential equation that describes this balance between convection and diffusion (and, in some cases, permeation) and then solving it subject to appropriate boundary conditions, an expression can be obtained for the ratio of the solute concentration at the membrane interface Cₘ relative to its concentration C_{b} in the bulk. For the particular case of a completely rejected solute (i.e., Rᵢ=1; Cₚ=0) with diffusivity D, this expression takes the form:
Cₘ/C_{b}=exp [(Jᵥ·δ)/D] Eq. 6

The grouping within the square brackets in Equation 6 will be recognized as the Peclet number Pe, i.e., the ratio of convective to diffusive fluxes, as discussed in considerable detail above. Substitution in Equation 6 leads to the simplified expression
Cₘ/C_{b}=exp (Pe) Eq. 7

In a typical separation application involving UF, δ generally refers to the effective thickness of the stagnant film or fluid boundary layer in contact with the surface of the membrane. In the present context, however, δ represents the effective thickness of the microchannel/microvessel element of the CMRA; that is, δ may be viewed either as the height of the microchannels or, alternatively, as the depth of the microwells in a confined membrane reactor array. Appropriate and straightforward modifications to the equations can be made where it is necessary to take into account the tortuosity and/or void volume of any structure (e.g., CMRA microchannels) that may reside atop the membrane surface. Inspection of the form of Equation 6 shows that the solute concentration within the microchannels or microwells of a polarized CMRA structure increases exponentially with distance as the surface of the porous membrane is approached.

The factor Cₘ/ C_{b} is termed the “concentration polarization modulus,” and it is readily calculated from the Peclet number Pe that quantifies the relative rates of the competing convective and diffusive transport processes. Typical values of the concentration polarization modulus are given in the following table for the limiting case of a completely rejected solute (i.e., Rᵢ=1):

| Microchannel Peclet | Concentration Polarization |
|---|---|
| Number Pe | Modulus (Cₘ/C_{b}) |
| 0.10 | 1.11 |
| 0.20 | 1.22 |
| 0.50 | 1.65 |
| 1.0 | 2.72 |
| 1.5 | 4.48 |
| 2.0 | 7.39 |
| 3.0 | 20.1 |
| 4.0 | 54.6 |
| 5.0 | 148. |
| 10.0 | 22,030 |

For the sake of definiteness, consider a small, completely rejected protein (e.g., ribonuclease) with a diffusion coefficient D of about 1·10⁻⁶ cm²/s being swept by convection into a CMRA microchannel or microwell with a length or depth δ of 10 μm at a microchannel flow velocity (alternately, V or Jᵥ) of 0.004 cm/s. The calculated Peclet number Pe corresponding to this situation is 4, and the resulting concentration polarization modulus Cₘ/ C_{b} is estimated to be of order 55—that is, the solute concentration at the membrane surface (i.e., at the bottom of the CMRA microchannel or well) will be 55 times higher than that in the bulk fluid at the top or mouth of the microchannel or well.

As noted previously, the solute concentration will vary exponentially with distance as measured from the top of a microchannel or mouth of a microwell, with the steepest gradient occurring at the base of the channel or well. More particularly, the concentration Cₓ of a polarized macrosolute at any point x along the length of a CMRA microchannel or microwell can be obtained by substituting the positional value x for the depth or thickness parameter δ in Equation 6:
Cₓ/C_{b}=exp [(Jᵥ·x)/D] Eq. 8

For the particular example of the small protein considered in the immediately preceding paragraph, the following local concentration factors Cₓ/C_{b} are obtained as a function of distance x from the top or mouth of the microchannel or well:

| Depth x (μm) | Cₓ/C_{b} |
|---|---|
| 0.0 | 1.0 |
| | (at top or mouth of microvessel) |
| 2.0 | 2.2 |
| 4.0 | 5.0 |
| 6.0 | 12.2 |
| 8.0 | 24.5 |
| 10.0 | 54.6 |
| | (at base of well or microchannel) |

The average concentration of a concentration-polarized solute within a CMRA microchannel or microwell is readily calculated by integrating Equation 8 with respect to the position coordinate x over the interval from x=0 to x=δ or L.

Lower-molecular-weight solutes that are incompletely rejected by the ultrafiltration membrane (i.e., Rᵢ<1) will also experience concentration polarization, albeit to a lesser extent. The concentration polarization moduli of such solutes will be smaller, as a consequence of their permeation or “leakage” across the ultrafilter. Although the mathematical equations that describe this situation are more tedious, they are known in the art and straightforward to solve and use.

Thus, while concentration polarization is normally considered a problem during conventional ultrafiltration because the increased concentration of retained molecules at the membrane surface increases the resistance to flow through it, the phenomenon is advantageous in the CMRA, where it is used specifically to create an elevated concentration of high-molecular-weight reagents inside the microchannels or microvessels. The increased concentration of macromolecules is then maintained by continued flow through the microchannels/microvessels and across the ultrafiltration membrane.

The maximum concentration of a molecule that is attainable in a CMRA—by concentration polarization or other means—is set by the solubility limit for that molecule. This limit is generally met at lower concentrations, the larger the molecule at hand (at least when solubility is expressed on a molar basis). When the solubility limit is exceeded, molecules—and especially high-molecular-weight biomolecules—will tend to come out of solution in the form of aggregates or gels, and where concentration polarization is the means by which high local concentrations are obtained, molecules will be deposited adjacent to the surface of the ultrafiltration membrane at a concentration corresponding to their solubility limit or gel concentration C_{g}. Formation of a gel is by no means required in the operation of a CMRA, but the process can be used to advantage, especially when particularly high local concentrations of molecules are desired. For many macromolecular solutions, gel concentrations C_{g} average around 25 wt % (with a range of from about 5% to 50%), whereas colloidal dispersions are characterized by C_{g} values that average about 65% (with a range from 50% to 75%). Once a gel layer forms atop an ultrafiltration membrane, the hydraulic permeability of the gel layer itself (rather than the intrinsic hydraulic permeability P of the UF membrane) can control the transmembrane flux Jᵥ.

Manipulation of the concentration of reaction participants by concentration polarization within a CMRA can be used to alter the phase or physical state of molecules in the system to advantage. For instance, if molecules in the gel state remain active (e.g., if an enzyme retains its bioactivity when precipitated in the form of a gel), then gel formation provides a means for obtaining very high local concentrations of that molecule within the microvessels or microwells of a CMRA. Furthermore, molecules that have precipitated into a gel are less subject to diffusional motion; indeed, the processes of gel relaxation and resolubilization can be quite slow—even irreversible—under certain circumstances. Thus, once macromolecules have been deposited in the form of gel layers by the application of convective flows sufficiently large as to cause severe concentration polarization and local concentrations that exceed the gel point concentration C_{g}, it is reasonable to expect that such molecules will tend to remain in the microchannels or microwells of a CMRA even when the convective flow rate through the microchannels or microwells is substantially reduced or even stopped. It may further be noted that molecules that form macromolecular complexes (e.g. multi-subunit proteins and certain polymers) can be added in bulk solution at concentrations that are too low for molecular association. Subsequently, however, they can be concentrated by the method of the present invention in the CMRA microchannels such that, e.g., polymers polymerize or multi-subunit proteins assemble.

Macromolecules can thus be maintained at elevated concentrations inside CMRA microchannels without having to attach said macromolecules to the walls of the microchannel or to some other solid-phase support; that is, the macromolecules remain localized in the solution phase (or perhaps the gel phase) without the need for covalent attachment to a solid-phase support. This is advantageous because many enzymes lose activity or exhibit decreased activity when covalently bound or otherwise associated with a surface (Bickerstaff, 1997). An additional advantage is that the macromolecule “localization” (cf. immobilization) method of the present invention is generic—i.e., it functions in substantially the same manner for all macrosolutes—rather than being macromolecule-specific, a drawback of many covalent immobilization protocols.

In certain reaction systems of interest (e.g., DNA analysis by pyrosequencing, as discussed in more detail below), it may be necessary to avoid covalently immobilizing certain macromolecular reagents altogether. The DNA polymerase used in pyrosequencing is a case in point. It is believed that DNA polymerase should retain at least a certain degree of mobility if it is to function optimally. As a consequence, this particular enzyme must normally be treated as a consumable reagent in pyrosequencing, since it is not desirable to covalently immobilize it and reuse it in subsequent pyrosequencing steps. However, the present invention provides means for localizing this macromolecular reagent within the microchannels or microvessels of a CMRA without having to covalently immobilize it.

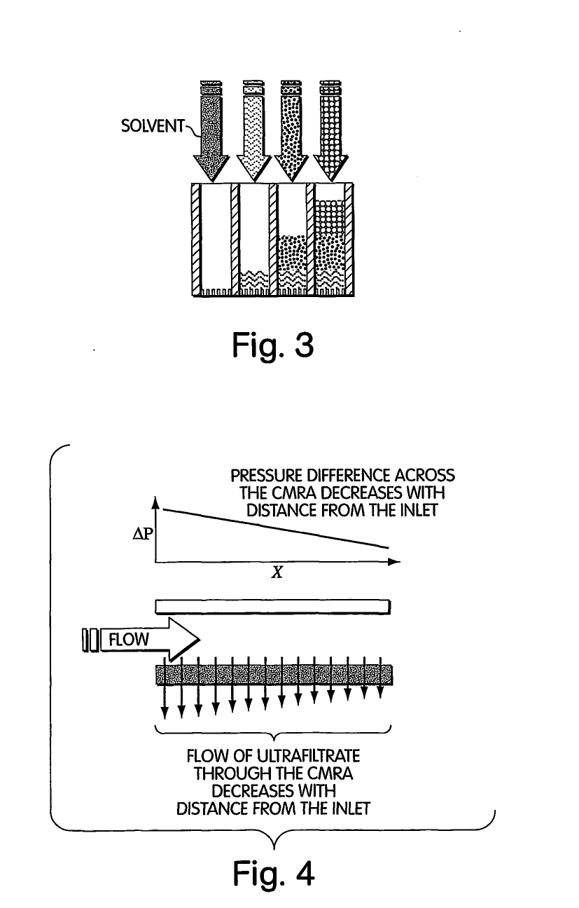

Macromolecules (e.g., enzymes) can be added to CMRA microchannels or microvessels in a sequential manner, thereby creating a microscopic stacked column (see FIG. 3). This permits sequential processing of reactants/substrates and their products in the channel, with products produced upstream being made available as reactants for downstream processing steps.

Once macromolecules have been concentrated and deposited in the microchannels or microwells of a CMRA by methods disclosed above, other reaction participants (e.g. reactants) can be added. If the molecules are small enough to pass through the ultrafiltration membrane substantially unimpeded, then their local concentration within the microchannels or microwells will be unaltered by the filtration process. Considering for the moment only the two limiting cases of Rᵢ=1 and Rᵢ=0, it is seen that molecules that are swept into the microchannels or microwells of a CMRA will experience either one of two fates: either a molecule will be concentrated and localized by ultrafiltration, or it will pass through CMRA and emerge in the permeate or “ultrafiltrate”. To simplify later descriptions, in what follows the word “pack” means to concentrate a molecule into the CMRA by concentration polarization, while the word “flow” means to generate bulk flow through the microchannel, carrying molecules into the CMRA and out in the ultrafiltrate without appreciable concentration within the CMRA.

As an aside, it should be noted that if the porous filter of the CMRA is capable of capturing particles in suspension (even if it is incapable of rejecting macromolecules in solution), then in a similar manner such particles will simply stack up next to the membrane in the form of a filter cake. In applications where certain of the reaction participants are immobilized on beads or other particulate supports, membranes other than ultrafilters—e.g., various microfiltration (MF) membranes known in the art—may be suitable for the practice of the present invention (Eykamp, 1995). The fundamental requirement is that the effective pore size of the membrane be comparable to or smaller than the diameter of the particles that one desires to retain.

Clearly, molecules entrapped and concentrated in the microchannels or microwells of a CMRA may still undergo diffusional motion to a certain extent. While molecules that have precipitated into a gel layer will exhibit decreased mobility—and perhaps substantially decreased mobility—it is still possible for them to return to solution and thus become free to diffuse, in the event that their concentration falls below the solubility or gel limit C_{g}. Referring to the stacked column configuration described above, it is evident that the order of the stack may be lost over time if the loaded macromolecules are subsequently able to diffuse at significant rates. To prevent this disordering, molecules can be tied together with polymers; for example, biotinylated macromolecules can be tied together with streptavidin-conjugated linear dextrans (e.g., 2M Dalton linear dextran/streptavidin conjugate, product number F071100-1, Amdex A/S, Denmark) or with one of any number of chemical crosslinkers. The crosslinker can be added after the proteins have been deposited within the CMRA in order to prevent premature crosslinking and aggregation during the loading step. Alternatively, biotinylated molecules and biotinylated linear dextran can be added concurrently, and then tied together by the subsequent addition of avidin (molecular weight ˜60 kD). Similarly, photoreactive crosslinkers can be added and then activated with light after other macromolecular species have been loaded into the microchannels or microwells of the CMRA.

As noted above, small molecules that would not typically be retained by ultrafilters can be attached to larger molecules (e.g. dextran or proteins such as albumin) or even to particles (e.g. polystyrene beads or colloidal gold, including porous beads such as those manufactured by Dynal, Inc.) to enhance their utility in connection with the present invention. Colloidal particles and microparticles will diffuse at much lower if not negligible rates as compared to microsolutes and macrosolutes. By attaching small molecules that would otherwise pass through the CMRA to larger macromolecules or particles, these smaller molecules can be retained in the microchannels or microwells of a CMRA.

It should further be noted that the present invention permits one to selectively manipulate the degree to which different reaction participants experience concentration polarization within the microchannels or microwells of a CMRA. Smaller molecules have larger diffusion coefficients, and so they will be characterized by smaller Peclet numbers; thus, the relative significance of the diffusional component of transport of these smaller molecules vis-a-vis the convective component will be greater for smaller molecules than it will be for larger ones. This provides a degree of freedom in the design and operation of these systems.

For example, the convective flow rate can be modulated to manipulate the Peclet number of various species selectively. At any flow rate V or Jᵥ across the CMRA, the smallest species (Rᵢ=0) will not be rejected at all by the porous membrane filter and will experience no concentration polarization. However, at a given flowrate small macromolecules with intermediate diffusivities may experience “intermediate” degrees of concentration polarization, while large macromolecules with small diffusivities will encounter “strong” polarization. If, then, one increases the flow rate and hence the Peclet number Pe for the smaller of two macromolecules, the extent of polarization of this molecule may be increased from “intermediate” to “strong.” If instead the flow rate and Pe are decreased, the degree of polarization of this smaller macromolecule may be reduced from “intermediate” to “low.” It should be noted, however, that at all three flow rate conditions, the smallest, unrejected solute will experience no polarization, while the larger of the two macromolecules will be strongly polarized.

As an example, reference to the above table shows that flow conditions could readily be chosen such that a small macromolecule characterized by a Peclet number Pe of 0.5 might exhibit a small concentration polarization modulus Cₘ/C_{b} of 1.65 at a first flow rate. However, increasing the flow rate 10-fold would result in a 10-fold increase in the Peclet number Pe to 5.0—and a substantial, 90-fold increase in the polarization modulus Cₘ/C_{b} to a relatively large value of 148. All this time, however, a much larger macromolecule that was strongly polarized to begin with at the lower flowrate (and perhaps even deposited as a gel) would remain strongly polarized at the higher flowrate.

Alternatively, flow could be slowed to such a degree that smaller molecules with larger diffusion coefficients (but with Rᵢ values greater than zero) would be permitted to diffuse throughout the microreactor volume—in the extreme, small molecules might even be permitted to diffuse upstream and out of the microreactor—while at the same time larger molecules with smaller diffusivities would experience significant concentration polarization. Flow speed could then be increased to restore concentration polarization for both species, while smaller molecules, unrejected or poorly rejected by the porous filter or membrane, were swept past larger molecules. At the extreme, convective flow could be stopped altogether, causing all molecular transport to occur by diffusion.

Manipulation of the Peclet number in this manner thus permits sequential processing steps—e.g., macromolecule packing, reactants supply, chemical conversion, and product removal in the ultrafiltrate—to be conducted in a highly controlled and advantageous manner. These steps can be performed in a constant-flow-rate system, or each step can be performed with different and time-varying flow speeds.

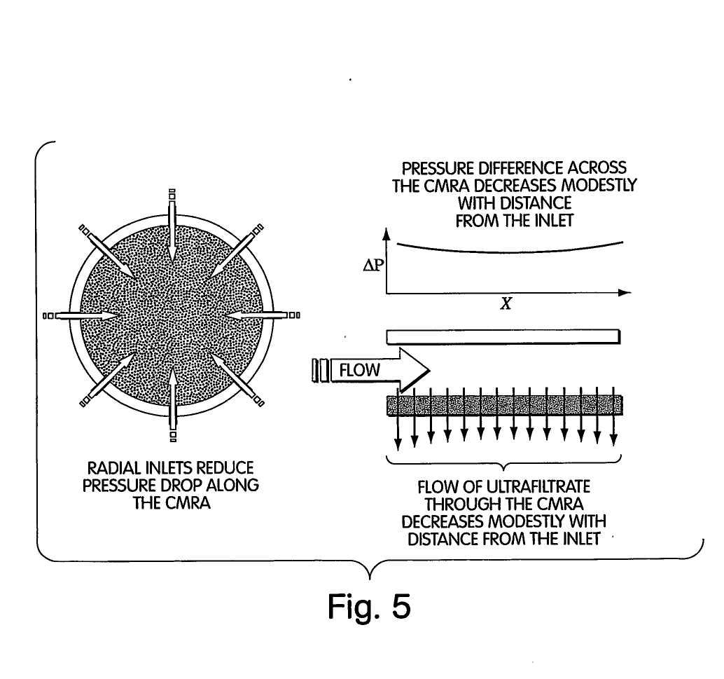

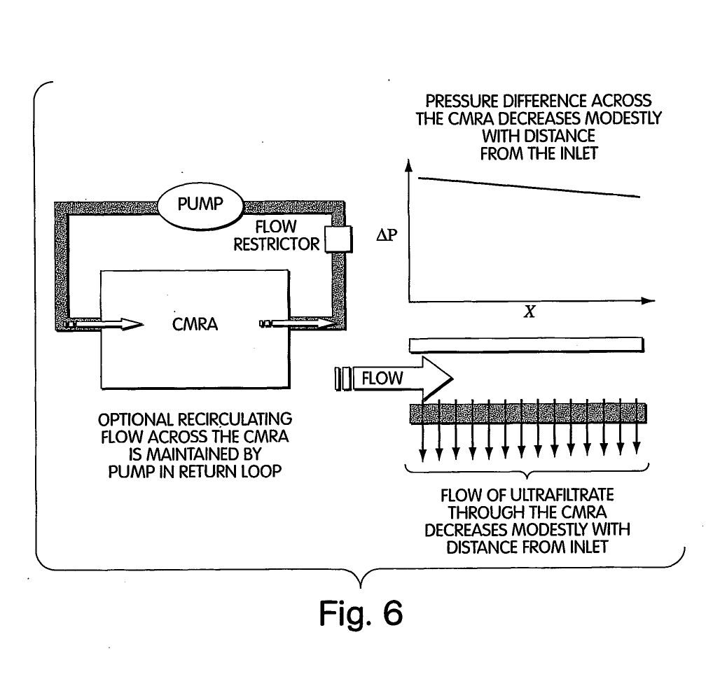

Controlling the pressure difference across the CMRA can, in principle, pose some minor problems if the fluid enters the flow compartment atop the CMRA via a plenum to one side of it. In this situation, there will be a pressure drop along the length of the fluid compartment atop and parallel to the CMRA due to viscous nature of the flows both parallel to and through the substantially 2-D confined membrane reactor array (FIG. 4). This pressure variation along the CMRA may, in extreme cases, cause the pressure difference across the CMRA (i.e. the pressure difference driving flow through it) to vary somewhat with distance, with the highest trans-CMRA pressure drop existing near the entrance plenum and the lowest pressure drop prevailing at the opposite end of the flow compartment. This effect can be reduced by introducing fluid via multiple inlets located along the sides or the circumference of the CMRA (see, for example, FIG. 5, where optional means to permit excess fluid to be withdrawn through a hole at the center of the CMRA may be provided). Alternatively, a back-pressure regulator, valve, or other flow restrictor may be introduced in the flow stream downstream of the CMRA, said regulator or valve introducing a controlling pressure drop that is arranged to be large as compared to the pressure drop within the CMRA flow compartment. In this manner, the fluid pressure within said flow compartment is increased—and the end-to-end variation in pressure drop across the CMRA itself is minimized (FIG. 6). A fluid recirculation loop may optionally be provided. By these and other means, the potential variation in pressure drop parallel to the plane of the CMRA can be arranged to be only a small fraction of the pressure drop normal to and across the CMRA.

In a preferred embodiment of a CMRA, the microreactor element comprising an array of microchannels or microwells is defined as a fiberoptic reactor array plate similar to that described in US patent 6,274,320. In that patent the fiber optic reactor array is formed by etching one end a fused fiber optic bundle to forms wells. In the context of one CMRA embodiment of this invention, the fiber optic array plate is etched completely through the entire width of the plate so that there is a series of open channels running from the top face of the plate through to the bottom face of the plate. A porous filter element is then be contacted to one face of such an etched fiber optic in order to form the CMRA.

In this embodiment, the microreactor array component is formed from a plate comprised of a fused fiber optic bundle. In such a fiber optic plate typically the distance between the top surface or face and the bottom surface or face is no greater than 5 cm, preferably no greater than 2 cm, and most preferably between I cm and 1 mm thick.

A series of microchannels extending from the top face to the botton face are created by treating the fiber optic plate, e.g., with acid. Each channel can form a reaction chamber. (see e.g., Walt, et al., 1996. Anal. Chem. 70: 1888).

The CMRA array typically contains more than 1,000 reaction chambers, preferably more than 400,000, more preferably between 400,000 and 20,000,000, and most preferably between 1,000,000 and 16,000,000 cavities or reaction chambers. When a fiber optic plate is used as the microreactor element, the shape of each reaction chamber (from a top view) is frequently substantially hexagonal, but the reaction chambers may also be cylindrical. In some embodiments, each reaction chamber has a smooth wall surface, however, we contemplate that each reaction chamber may also have at least one irregular wall surface. The array is typically constructed to have reaction chambers with a center-to-center spacing between 5 to 200 μm, preferably between 10 to 150 μm, most preferably between 50 to 100 μm. In one embodiment, we contemplate that each reaction chamber has a width in at least one dimension of between 0.3 μm and 100 μm, preferably between 0.3 μm and 20 μm, most preferably between 0.3 μm and 10 μm. In a separate embodiment, we contemplate larger reaction chambers, preferably having a width in at least one dimension of between 20 μm and 70 μm.

UMRAs

Yet another technique for creating a membrane-based microreactor array eliminates the need for discrete microchannels or microwells altogether. This membrane reactor array is comprised simply of a porous filter against which molecules are concentrated by concentration polarization (or by which particles are packed by filtration) just as described above. In this instance, however, some of the concentration-polarized molecules may be made to form a continuous 2-D layer atop the rejecting layer of the porous membrane, while other reaction participants are deposited and/or otherwise immobilized or localized at discrete, independent sites within or atop the structure. Such membrane reactor arrays are referred to hereinafter as “unconfined membrane reactor arrays” or UMRAs.

Discrete reactions can be made to occur in discrete locations by “seeding” the surface of this membrane array with individual molecules or particles that initiate the reaction. For example, a catalyst could be added by pipetting or “spotting” small regions of the surface with a dilute solution thereof. Alternatively, a catalyst could be added in bulk solution at such low concentration such that, upon filtration onto the surface, the density of catalyst deposited in the plane of the UMRA would be reasonably high but discontinuous, i.e., the catalyst might be “dotted” over the surface of the 2-D layer. In yet another embodiment, the catalyst (e.g., an enzyme) might be bound to a particulate or colloidal support (e.g., by covalent immobilization); a dilute suspension thereof would then be filtered through the UMRA, causing deposition of catalyst beads or particles at discrete sites on the surface.

The UMRA array typically contains more than 1,000 reaction chambers, preferably more than 400,000, more preferably between 400,000 and 20,000,000, and most preferably between 1,000,000 and 16,000,000 cavities or reaction chambers. When a fiber optic plate is used as the microreactor element, the shape of each reaction chamber (from a top view) is frequently substantially hexagonal, but the reaction chambers may also be cylindrical. In some embodiments, each reaction chamber has a smooth wall surface, however, we contemplate that each reaction chamber may also have at least one irregular wall surface. The array is typically constructed to have reaction chambers with a center-to-center spacing between 5 to 200 μm, preferably between 10 to 150 μm, most preferably between 50 to 100 μm. In one embodiment, we contemplate that each reaction chamber has a width in at least one dimension of between 0.3 μm and 100 μm, preferably between 0.3 μm and 20 μm, most preferably between 0.3 μm and 10 μm. In a separate embodiment, we contemplate larger reaction chambers, preferably having a width in at least one dimension of between 20 μm and 70 μm.

In still other instances, it may be advantageous to deposit a particular reactant molecule (e.g., an oligonucleotide or construct thereof) at discrete sites on the surface of a UMRA (e.g., for pyrosequencing). Again, this may be accomplished either by pipetting solutions thereof onto the surface of the UMRA, by ultrafiltering extremely dilute solutions of reactants through the UMRA, or, preferably, by immobilizing said reactants on particulate or colloidal supports and then depositing these onto the UMRA surface by ultrafiltration.

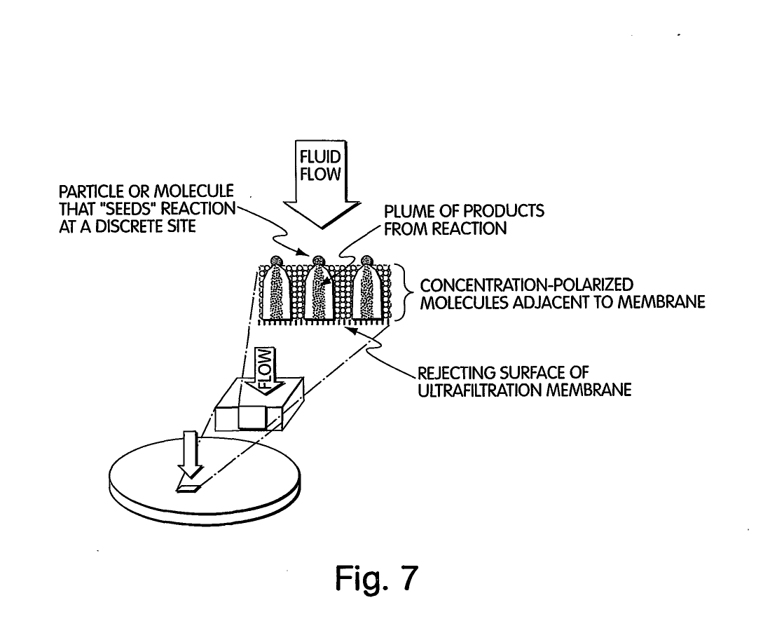

A distinguishing feature of the UMRA relative to the CMRA is that lateral diffusion of molecules in the UMRA is not confined by the walls of a microchannel or microvessel as is the case with the CMRA. To restrict the impact of lateral diffusion of molecules in a UMRA, molecules are swept through the UMRA and into the filtrate before their lateral transport can proceed to an extent such that it becomes problematic (FIG. 7). (Note that for the sake of simplicity, FIG. 7 shows only the rejecting surface or “skin layer” of the ultrafilter; the substrate region and other membrane supports, if present—have been omitted for the sake of clarity.) Again, manipulation of the flow velocity (and species Peclet numbers) permits control of the extent of lateral transport as well as the residence times of molecules within the UMRA. The flow rate can be slowed, thus increasing residence time and the extent of lateral transport; or the flow rate can be increased, with a concomitant decrease in residence time and lateral transport. It should be noted that asymmetric ultrafiltration membranes of the “integral composite” type may be employed in a UMRA in either orientation—that is, with either their rejecting layer “up” (and spongy substrate region “down”) or vice versa.

In addition to its having a porous ultrafiltration membrane component, a UMRA can also employ more porous and substantially non-rejecting filters (or functionally equivalent matrices) either upstream or downstream of the ultrafilter and either placed against and/or attached to the ultrafiltration membrane itself (FIG. 8). This non-selective secondary filter, characterized by its much larger pore sizes, can be used to provide mechanical support to the permselective ultrafiltration membrane. It can also be used as a mesh or matrix that provides some mechanical support and protection to the concentrated molecules at the membrane surface, thereby stabilizing this layer of molecules. In particular, the provision of such a mesh or matrix can shield the surface layer of concentrated macromolecules and particles from the shearing action of any tangential flow that may be directed along the upper surface of the UMRA. When asymmetric, integrally composite UF membranes of the type mentioned in the preceding paragraph are employed, their spongy and relatively thick substrate region may conveniently serve as a stabilizing matrix if the UF membrane is oriented with the skinned rejecting surface “down”.

A UMRA can be assembled and operated substantially in the manner of a CMRA. Species Peclet numbers can be manipulated by controlling the flow rate, and different macromolecules and/or particles can be packed in sequence to create a stacked microreactor. Molecules can be added at concentrations below their K_{d} or C_{g} values, and then concentrated above their K_{d} or C_{g} values by concentration polarization at the filter surface. Molecules can be attached to other molecules, to polymers, or to particles. Molecules can also be enmeshed within polymers.

Many different types of reactions can be performed in a CMRA or UMRA. In one embodiment, each cavity or reaction chamber of the array contains reagents for analyzing a nucleic acid or protein. Typically those reaction chambers that contain a nucleic acid (not all reaction chambers in the array are required to) contain only a single species of nucleic acid (i.e., a single sequence that is of interest). There may be a single copy of this species of nucleic acid in any particular reaction chamber, or they may be multiple copies. It is generally preferred that a reaction chamber contain at least 100 copies of a nucleic acid sequence, preferably at least 100,000 copies, and most preferably between 100,000 to 1,000,000 copies of the nucleic acid. In one embodiment the nucleic acid species is amplified to provide the desired number of copies using PCR, RCA, ligase chain reaction, other isothermal amplification, or other conventional means of nucleic acid amplification. In one embodimant, the nucleic acid is single stranded. In other embodiments the single stranded DNA is a concatamer with each copy covalently linked end to end.

The nucleic acid may be immobilized in the reaction chamber, either by attachment to the chamber itself or by attachment to a mobile solid support that is delivered to the chamber. A bioactive agent could be delivered to the array, by dispersing over the array a plurality of mobile solid supports, each mobile solid support having at least one reagent immobilized thereon, wherein the reagent is suitable for use in a nucleic acid sequencing reaction.

The array can also include a population of mobile solid supports disposed in the reaction chambers, each mobile solid support having one or more bioactive agents (such as a nucleic acid or a sequencing enzyme) attached thereto. The diameter of each mobile solid support can vary, we prefer the diameter of the mobile solid support to be between 0.01 to 0.1 times the width of each cavity. Not every reaction chamber need contain one or more mobile solid supports. There are three contemplated embodiments; one where at least 5% to 20% of of the reaction chambers can have a mobile solid support having at least one reagent immobilized thereon; a second embodiment where 20% to 60% of the reaction chambers can have a mobile solid support having at least one reagent immobilized thereon; and a third embodiment where 50% to 100% of the reaction chambers can have a mobile solid support having at least one reagent immobilized thereon.

The mobile solid support typically has at least one reagent immobilized thereon. For the embodiments relating to pyrosequencing reactions or more generally to ATP detection, the reagent may be a polypeptide with sulfurylase or luciferase activity, or both. Alternatively, enzymes such as hypoxanthine phosphoribosyltransferase, xanthine oxidase, uricase or peroxidase could be utilized (e.g., Jansson and Jansson (2002), incorporated herein by reference). The mobile solid supports can be used in methods for dispersing over the array a plurality of mobile solid supports having one or more nucleic sequences or proteins or enzymes immobilized thereon.

In another aspect, the invention involves an apparatus for simultaneously monitoring the array of reaction chambers for light generation, indicating that a reaction is taking place at a particular site. In this embodiment, the reaction chambers are sensors, adapted to contain analytes and an enzymatic or fluorescent means for generating light in the reaction chambers. In this embodiment of the invention, the sensor is suitable for use in a biochemical or cell-based assay. The apparatus also includes an optically sensitive device arranged so that in use the light from a particular reaction chamber would impinge upon a particular predetermined region of the optically sensitive device, as well as means for determining the light level impinging upon each of the predetermined regions and means to record the variation of the light level with time for each of the reaction chamber.

In one specific embodiment, the instrument includes a light detection means having a light capture means and a second fiber optic bundle for transmitting light to the light detecting means. We contemplate one light capture means to be a CCD camera. The second fiber optic bundle is typically in optical contact with the array, such that light generated in an individual reaction chamber is captured by a separate fiber or groups of separate fibers of the second fiber optic bundle for transmission to the light capture means.

The invention provides an apparatus for simultaneously monitoring an array of reaction chambers for light indicating that a reaction is taking place at a particular site. The reaction event, e.g., photons generated by luciferase, may be detected and quantified using a variety of detection apparatuses, e.g., a photomultiplier tube, a CCD, CMOS, absorbance photometer, a luminometer, charge injection device (CID), or other solid state detector, as well as the apparatuses described herein. In a preferred embodiment, the quantitation of the emitted photons is accomplished by the use of a CCD camera fitted with a fused fiber optic bundle. In another preferred embodiment, the quantitation of the emitted photons is accomplished by the use of a CCD camera fitted with a microchannel plate intensifier. A back-thinned CCD can be used to increase sensitivity. CCD detectors are described in, e.g., Bronks, et al., 1995. Anal. Chem. 65: 2750-2757.

An exemplary CCD system is a Spectral Instruments, Inc. (Tucson, Ariz.) Series 600 4-port camera with a Lockheed-Martin LM485 CCD chip and a 1-1 fiber optic connector (bundle) with 6-8 μm individual fiber diameters. This system has 4096×4096, or greater than 16 million pixels and has a quantum efficiency ranging from 10% to >40%. Thus, depending on wavelength, as much as 40% of the photons imaged onto the CCD sensor are converted to detectable electrons.

In other embodiments, a fluorescent moiety can be used as a label and the detection of a reaction event can be carried out using a confocal scanning microscope to scan the surface of an array with a laser or other techniques such as scanning near-field optical microscopy (SNOM) are available which are capable of smaller optical resolution, thereby allowing the use of “more dense” arrays. For example, using SNOM, individual polynucleotides may be distinguished when separated by a distance of less than 100 nm, e.g., 10 nm×10 nm. Additionally, scanning tunneling microscopy (Binning et al., Helvetica Physica Acta, 55:726-735, 1982) and atomic force microscopy (Hanswa et al., Annu Rev Biophys Biomol Struct, 23:115-139, 1994) can be used.

Manufacture of CMRAs and UMRAs and Uses Thereof

The invention provides a CMRA and UMRA which are both an array comprising densely packed, independent chemical reactions The reaction site of the CMRA is a microreactor vessel or a microwell. The invention also includes method for making the CMRA and UMRA dense array of discrete reaction sites. The invention also provides a method for charging a microreactor with reaction participants, the method comprising, effecting convective flow of fluid normal to the plane of and through the array of reaction sites. In a preferred embodiment of both the CMRA and UMRA, the reactants may be either attached or unattached to a solid support. The attached reactants are covalently bound to a solid support.

The invention also includes a method for efficiently supplying relatively lower molecular weight reagents and reactants to discrete reaction sites. The CMRA itself comprises a microreactor comprising a microchannel with an ultrafiltration membrane at one end. Specifically, the CMRA comprises a series of microchannels; a concentration polarization to create a packed column of molecules; a sequential packing of molecules via the concentration polarization to create stacked columns; and a flow of reagents through a packed column. In a preferred embodiment of the CMRA , each array is an independent chemical reactor.

The invention also provides a method of generating a CMRA, the method comprising the steps of: (a) flowing of reagents through a packed column/CMRA for sequential processing of chemicals; (b) adding random fragments of DNA, obtaining about one fragment per microchannel, by filtration of a mixture onto a CMRA; and (c) concentrating molecules into a gel inside the microchannels of a CMRA. Molecules are added to the CMRA below the K_{d} inside a microchannel, permitting polymerization and assembly of molecules only inside the microchannels. Crosslinkers can be added to decrease the diffusional mobility of molecules in a microchannel. Furthermore, polymers can also be added that enmesh molecules inside a microchannel, as well as smaller molecules attached to larger molecules or to larger particles or to beads to decrease the diffusional mobility of the smaller molecule. Smaller molecules, that would otherwise pass through the filter, attached to larger molecules or to larger particles or to beads to retain the smaller molecules in the microchannel of a CMRA can be added as well.

The CMRA is generated by using Anopore membranes. More specifically, the CMRA is fabricated by bonding an ultrafiltration membrane to microfabricated array of microchannels. Fluid inlets are then radially distributed to equalize pressure across the membrane, reducing variation pressure over the CMRA. An ultrafiltration membrane, without the microchannels, is then used to create a dense 2-D array of chemical reactions (“unconfined membrane reactor array” or UMRA) in which reactions are seeded by filtering a catalyst or reactant or enzyme onto the filter surface and whereby convective flow washes away laterally diffusing molecules before they contaminate adjacent reactions. Concentration polarization is necessary to create the packed columns of molecules for the CMRA and UMRA followed by the sequential packing of molecules via concentration polarization to create stacked columns. Reagents are then flowed through a packed column of the CMRA or UMRA for sequential processing of chemicals. The ultrafiltration membrane is then bonded to a second, more porous membrane to provide mechanical support to the molecules concentrated by concentration polarization. The membrane is a Molecular/Por membrane (Spectrum Labs).

The CMRA and UMRA have a multitude of uses including: PCR, as well as other DNA amplification techniques and DNA sequencing techniques, such as pyrosequencing. Both the CMRA and UMRA can be utilized to achieve highly parallel sequencing without separation of DNA fragments and associated sample prep. The CMRA and UMRA can also be used for combinatorial chemistry. For detection purposes, an array of photodetectors are utilized for monitoring light producing reactions within the CMRA or UMRA. In a preferred embodiment, the array of photodetectors is a CCD camera. Another method of detection of discrete reactions withinn the CMRA and UMRA is to monitor changes in light absorption as an indicator of a chemical reaction in a CMRA using an array of photodetectors.

Two examples are offered below as specifically contemplated uses for CMRAs and UMRAs, but these are meant only to be representative and should not be considered as the only applications or embodiments of the present invention.

Sequencing of DNA Via Pyrophosphate Detection

The methods and apparatuses described are generally useful for any application in which the identification of any particular nucleic acid sequence is desired. For example, the methods allow for identification of single nucleotide polymorphisms (SNPs), haplotypes involving multiple SNPs or other polymorphisms on a single chromosome, and transcript profiling. Other uses include sequencing of artificial DNA constructs to confirm or elicit their primary sequence, or to identify specific mutant clones from random mutagenesis screens, as well as to obtain the sequence of cDNA from single cells, whole tissues or organisms from any developmental stage or environmental circumstance in order to determine the gene expression profile from that specimen. In addition, the methods allow for the sequencing of PCR products and/or cloned DNA fragments of any size isolated from any source.

Sequencing of DNA by pyrophosphate detection (“pyrophosphate sequencing”) is described in various patents (Hyman, 1990, U.S. Pat. No. 4,971,903; Nyren et al., U.S. Pat. Nos. 6,210,891 and 6,258,568 and PCT Patent application WO98/13523; Hagerlid et al., 1999, WO99/66313; Rothberg, U.S. Pat. No. 6,274,320, WO01/20039) and publications (Hyman, 1988; Nyrén et al., 1993; Ronaghi et al., 1998, Jensen, 2002; Schuller, 2002). The contents of the foregoing patents, patent applications and publications cited here are incorporated herein by reference in their entireties. Pyrophosphate sequencing is a technique in which a complementary sequence is polymerized using an unknown sequence (the sequence to be determined) as the template. This is, thus, a type of sequencing technique known as “sequencing by synthesis”. Each time a new nucleotide is polymerized onto the growing complementary strand, a pyrophosphate (PPi) molecule is released. This release of pyrophosphate is then detected. Iterative addition of the four nucleotides (dATP, dCTP, dGTP, dTTP) or of analogs thereof (e.g., α-thio-dATP), accompanied by monitoring of the time and extent of pyrophosphate release, permits identification of the nucleotide that is incorporated into the growing complementary strand.

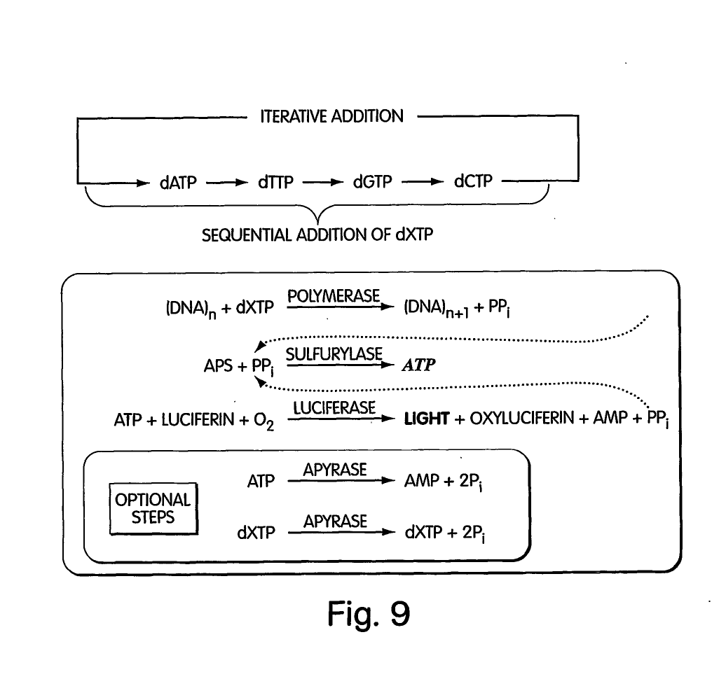

Pyrophosphate can be detected via a coupled reaction in which pyrophosphate is used to generate ATP from adenosine 5′-phosphosulfate (APS) through the action of the enzyme ATP sulfurylase (FIG. 9). The ATP is then detected photometrically via light released by the enzyme luciferase, for which ATP is a substrate. (It may be noted that dATP is added as one of the four nucleotides for sequencing by synthesis and that luciferase can use dATP as a substrate. To prevent light emission on addition of dATP for sequencing, a dATP analog such as α-thio-dATP is substituted for dATP as the nucleotide for sequencing. The α-thio-dATP molecule is incorporated into the growing DNA strand, but it is not a substrate for luciferase.)

Pyrophosphate sequencing can be performed in a CMRA or UMRA in several different ways. One such protocol follows:

(1) pack luciferase;

(2) pack ATP sulfurylase;

(3) pack the DNA whose sequence is to be determined (preferably, many copies of a single sequence) and DNA polymerase (e.g. Klenow fragment); and

(4) flow a mixture of dXTP, APS, and luciferin through the CMRA or UMRA, cycling through the four nucleotides (dCTP, dGTP, dTTP, α-thio-dATP) one at a time. It will be noted that these are all low-molecular weight molecules, so they will pass through the ultrafiltration membrane of the CMRA or UMRA (at least if the ultrafilter's MWCO is appropriately chosen) without said molecules undergoing appreciable concentration polarization.

The upstream-to-downstream flow of fluid into and through the CMRA or UMRA thus causes:

(a) addition of the appropriate dXTP by the polymerase and attendant production of PPᵢ in the region of the DNA being sequenced (with APS and luciferin flowing through passively);

(b) production of ATP from APS and PPᵢ when the latter are brought into contact with the sulfurylase enzyme (with luciferin flowing through passively); and

(c) production of light from ATP and luciferin in the vicinity of the luciferase enzyme.

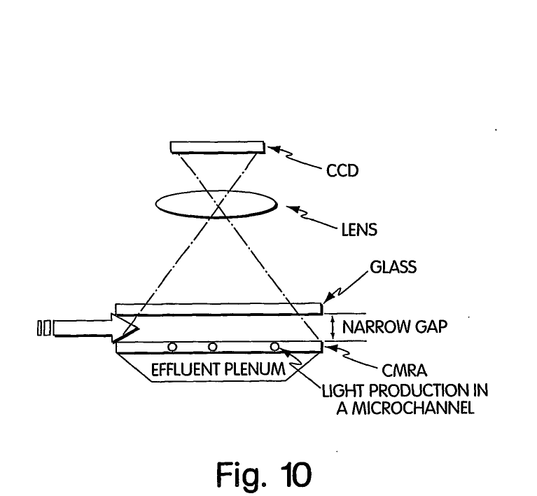

Light production is then monitored by a photodetector. For example, a CCD camera, optically coupled by a lens or other means to the CMRA or UMRA, is capable of monitoring light production simultaneously from many microchannels or discrete reaction sites (FIG. 10). CCD cameras are available with millions of pixels, or photodetectors, arranged in a 2-D array. Light originating from one microchannel, microwell, or discrete reaction site in or on a CMRA or UMRA can be made to strike one or a few pixels on the CCD. Thus, if each microchannel, microwell, or reaction site is arranged to contain and conduct an independent sequencing reaction, each reaction can be monitored by one (or at most a few) CCD elements or photodetectors. By using a CCD camera or other imaging means comprising millions of pixels, the progress of millions of independent sequencing reactions is simultaneously monitored.

Further, each microreactor vessel, well, or reaction site can be made to hold the amplification products from only a single strand of DNA, and if different wells hold the amplification products of different strands of DNA, then the simultaneous sequencing of millions of different strands of DNA is possible. The distribution of DNA to be sequenced can be accomplished in many ways, two of which follow:

(a) The amplification products of a single oligonucleotide strand are attached to a bead, and beads from many independent amplification reactions are combined and placed onto a CMRA or UMRA; or

(b) Many different strands of DNA are added in dilute concentration and applied to the CMRA or URMA such that many if not most microchannels, microvessels, or discrete reaction sites contain only a single strand of DNA. The DNA is then amplified within or upon the CMRA or URMA through one series of reactions, and then it is directly sequenced via addition of the reagents described above. One such technique (polymerase chain reaction or PCR) for amplification of DNA within the microchannels of a CMRA (or UMRA) is described below.

Delivery of the DNA to be sequenced and the enzymes and substrates necessary for pyrophosphate-based sequencing can be accomplished in a number of ways.

In a preferred embodiment, one or more reagents are delivered to the CMRA or UMRA immobilized or attached to a population of mobile solid supports, e.g., a bead or microsphere. The bead or microsphere need not be spherical, irregular shaped beads may be used. They are typically constructed from numerous substances, e.g., plastic, glass or ceramic and bead sizes ranging from nanometers to millimeters depending on the width of the reaction chamber. Preferably, the diameter of each mobile solid support can be between 0.01 and 0.1 times the width of each reaction chamber. Various bead chemistries can be used e.g., methylstyrene, polystyrene, acrylic polymer, latex, paramagnetic, thoria sol, carbon graphite and titanium dioxide. The construction or chemistry of the bead can be chosen to facilitate the attachment of the desired reagent.

In another embodiment, the bioactive agents are synthesized first, and then covalently attached to the beads. As is appreciated by someone skilled in the art, this will be done depending on the composition of the bioactive agents and the beads. The functionalization of solid support surfaces such as certain polymers with chemically reactive groups such as thiols, amines, carboxyls, etc. is generally known in the art. Accordingly, “blank” beads may be used that have surface chemistries that facilitate the attachment of the desired functionality by the user. Additional examples of these surface chemistries for blank beads include, but are not limited to, amino groups including aliphatic and aromatic amines, carboxylic acids, aldehydes, amides, chloromethyl groups, hydrazide, hydroxyl groups, sulfonates and sulfates.

These functional groups can be used to add any number of different candidate agents to the beads, generally using known chemistries. For example, candidate agents containing carbohydrates may be attached to an amino-functionalized support; the aldehyde of the carbohydrate is made using standard techniques, and then the aldehyde is reacted with an amino group on the surface. In an alternative embodiment, a sulfhydryl linker may be used. There are a number of sulfhydryl reactive linkers known in the art such as SPDP, maleimides, α-haloacetyls, and pyridyl disulfides (see for example the 1994 Pierce Chemical Company catalog, technical section on cross-linkers, pages 155-200, incorporated here by reference) which can be used to attach cysteine containing proteinaceous agents to the support. Alternatively, an amino group on the candidate agent may be used for attachment to an amino group on the surface. For example, a large number of stable bifunctional groups are well known in the art, including homobifunctional and heterobifunctional linkers (see Pierce Catalog and Handbook, pages 155-200).

In an additional embodiment, carboxyl groups (either from the surface or from the candidate agent) may be derivatized using well known linkers (see Pierce catalog). For example, carbodiimides activate carboxyl groups for attack by good nucleophiles such as amines (see Torchilin et al., Critical Rev. Thereapeutic Drug Carrier Systems, 7(4):275-308 (1991)). Proteinaceous candidate agents may also be attached using other techniques known in the art, for example for the attachment of antibodies to polymers; see Slinkin et al., Bioconj. Chem. 2:342-348 (1991); Torchilin et al., supra; Trubetskoy et al., Bioconj. Chem. 3:323-327 (1992); King et al., Cancer Res. 54:6176-6185 (1994); and Wilbur et al., Bioconjugate Chem. 5:220-235 (1994). It should be understood that the candidate agents may be attached in a variety of ways, including those listed above. Preferably, the manner of attachment does not significantly alter the functionality of the candidate agent; that is, the candidate agent should be attached in such a flexible manner as to allow its interaction with a target.

Specific techniques for immobilizing enzymes on beads are known in the prior art. In one case, NH₂ surface chemistry beads are used. Surface activation is achieved with a 2.5% glutaraldehyde in phosphate buffered saline (10 mM) providing a pH of 6.9 (138 mM NaCl, 2.7 mM KCl). This mixture is stirred on a stir bed for approximately 2 hours at room temperature. The beads are then rinsed with ultrapure water plus 0.01% Tween 20 (surfactant) −0.02%, and rinsed again with a pH 7.7 PBS plus 0.01% tween 20. Finally, the enzyme is added to the solution, preferably after being prefiltered using a 0.45 μm amicon micropure filter.

In some embodiments, the reagent immobilized to the mobile solid support can be a polypeptide with sulfurylase activity, a polypeptide with luciferase activity or a chimeric polypeptide having both sulfurylase and luciferase activity. In one embodiment, it can be a ATP sulfurylase and luciferase fusion protein. Since the product of the sulfurylase reaction is consumed by luciferase, proximity between these two enzymes may be achieved by covalently linking the two enzymes in the form of a fusion protein. In other embodiments, the reagent immobilized to the mobile solid support can be the nucleic acid whose sequence is to be determined or analyzed.

Many variations and alternative embodiments of the present invention as applied to DNA sequencing and other applications will be readily apparent and are considered to be within the scope of the present invention.

Polymerase Chain Reaction (PCR)

PCR can also be performed in an CMRA (or a UMRA). Participants in a PCR reaction include template DNA, primers, polymerase, and deoxynucleotides. Diffusion coefficients for some of these molecules follow:

| Molecule | D (10⁻⁵ cm²/s) |
|---|---|
| Taq Polymerase | 0.06* |
| DNA (1100mer, duplex) | 0.007** |

*assumed to be slightly less than ovalbumin, which has a lower molecular weight (45 kD for ovalbumin cf. 90 kD for Taq Polymerase); D for ovalbumin is 0.08 · 10⁻⁵ (Cussler, 1997).
**Liu et al. 2000

A DNA sequence to be amplified is packed into the CMRA, with as few as a single copy of a sequence per microchannel or microwell. Polymerase and primer are packed next, and nucleotides are added via flow. Thermal cycling then proceeds with heating of the CMRA by IR or other means of thermal control. Continuous flow of dXTP solution (and in some instances primer) through the CMRA ensures the retention of amplification products within the microchannels or microwells of the CMRA as the solution of dXTP (and perhaps primer) is continually added. The result is an independent array of PCR reactions conducted in independent CMRA microchannels or microwells, with each amplifying different DNA sequences.

It may be noted that other DNA amplification techniques can be performed in a similar manner. Such alternative techniques include bridge amplification onto a bead, rolling circle amplification (RCA) to form linear oligonucleotide concatamers, and hyperbranching amplification.

### EXAMPLES

### Example 1

Pyrophosphate-Based Sequencing in a UMRA Materials

Reagents. Sepharose beads are 30±10 um and can bind 1×10⁹ biotin molecules per bead (very high binding capacity). Sequences of Oligonucleotides used in PCR on the membrane; Cy3-labelled probe J (5′-[Cy3]ATCTCTGCCTACTAACCATGAAG-3′) (SEQ ID NO: 1), Biotinyalted probe (5′-RBiot(dT18) GTTTCTCTCCAGCCTCTCACCGA-3′) (SEQ ID NO:2), SsDNA template (5′-ATC TCT GCC TAC TAA CCA TGA AGA CAT GGT TGA CAC AGT GGA ATT TTA TTA TCT TAT CAC TCA GGA GAC TGA GAC AGG ATT GTC ATA AGT TTG AGA CTA GGT CGG TGA GAG GCT GGA GAG AAA C-3′) (SEQ ID NO:3), and Non-Biotinylated probe (5′-GTTTCTCTCCAGCCTCTCACCGA-3′) (SEQ ID NO:4), Seq1 5′-ACG TAA AAC CCC CCC CAA AAG CCC AAC CAC GTA CGT AAG CTG CAG CCA TCG TGT GAG GTC-3′ (SEQ ID NO:5), PRB1 5′-BS-GAC CTC ACA CGA TGG CTG CAG CTT-3′ (SEQ ID NO:6)

Preparation of Beads. Conjugation of biotinylated single stranded DNA probe to the streptavidin-bound Sepharose beads (Pharmacia Biotech, Uppsala, Sweden) was performed using the following protocol: 100 μl of Sepharose beads (1×10⁸ beads ml⁻¹) and 300 μl of binding wash buffer (as recommended by the manufacturer) were applied to a Microcon 100 (Amicon, Beverly, Mass.) membrane. The tube was spun at 5000 rpm for 6 min in a micro centrifuge. The beads were washed twice with 300 μl of binding wash buffer. The tube was inverted into a new tube and the beads were spun out of the membrane (1 min at 6000 rpm). The beads were allowed to settle and the supernatant was removed. 100 μl (1 pmol μl⁻¹) of biotinylated probe solution was added to the beads. The final sample volume was about 100 μl. The tube was placed on a rotator for 1 hr at room temperature to allow the conjugation of the biotinylated probe with the beads. After conjugation, the beads were washed 3 times with TE buffer as described above. The final bead volume was about 100 μl and stored at −20° C.

Methods

Loading enzymes on to the first membrane. Substrate consists of 300 μm D-Luciferin (Pierce, Rockford, Ill.), 4 μM of APS (Adenosin 5′-phosphosulfate sodium salt, Sigma), 4 mg/ml PVP (Polyvinyle Pyrolidone, Sigma), and 1 mM of DTT (Dithiothreitol, Sigma). 10 μl of recombinant luciferase (14.7 mg ml⁻¹, from Sigma) and 75 μl of ATP sulfurylase (1.3 mg ml⁻¹, from Sigma) were mixed in 1 ml of substrate. This enzyme mixture was pipetted onto an ultra filtration membrane (MWCO 30,000; Millipore Incorporation, Bedford, Mass.)) wetted with substrate. Suction was applied to trap the enzyme mixture into the microstructure of the membrane. The enzymes adsorbed onto the membrane were found to be stable for 18 hr at room temperature.

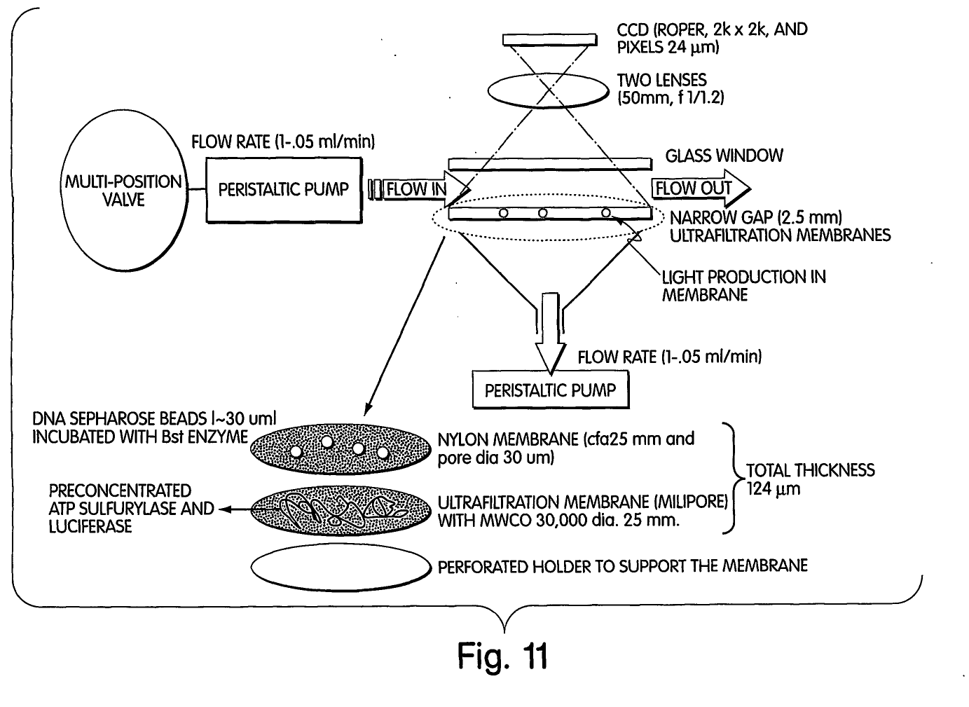

Loading DNA Sepharose beads on to the second membrane. 100 μl of Sepharose beads (1×10⁸ beads per ml) with bound DNA (3×10⁶ copies per bead) was diluted in substrate and applied to a second Nylon membrane (Rancho Dominguez, Calif.). The beads are 30±10 μm and the nylon membrane has a pore diameter 30 μm. Vacuum suction was applied to fix the beads onto the mesh of the membrane. The nylon membrane was then placed on top of the enzyme membrane. 80 μl of Bst DNA polymerase enzyme (8000 U μl⁻¹) was pipetted into the nylon membrane and the membrane was incubated for 30 min at room temperature. After incubation a glass window was placed on top of the membrane as shown in FIG. 11. The membrane holder was connected to Fluidic 1.1 consisting of a multiposition valve (from Valco Instruments (Houston, Tex.) and two peristaltic pumps (Instech Laboratories Inc., Plymouth Meeting, Pa.). During the substrate flow, the upper pump was running at a flow rate 0.5 ml min⁻¹ for 2 min and the lower pump was running constantly at a flow rate of 50 μl min⁻¹. During the flow of pyrophosphate (ppi) or nucleotide, the flow rate of the upper pump was 0.1 ml min⁻¹ for 2 min.

Imaging system. The imaging system consists of a CCD camera (Roper Scientific 2k×2k with a pixel size of 24 μm) and two lenses (50 mm, f 1/1.2). One lens collects the light produced at the result of the interaction of the ppi with the enzyme mixture and the other lens focus the light into the CCD camera. The acquisition time for all of the experiments was 1 sec. A run-off, in which dNTP's (6.5 μM of dGTP, 6.5 μM of dCTP, 6.5 μM of dTTP, and 50 μM of dATP-αs and for 2 min) were added to the DNA beads gave 5000 counts above background. Background was normally 160 counts.

Results

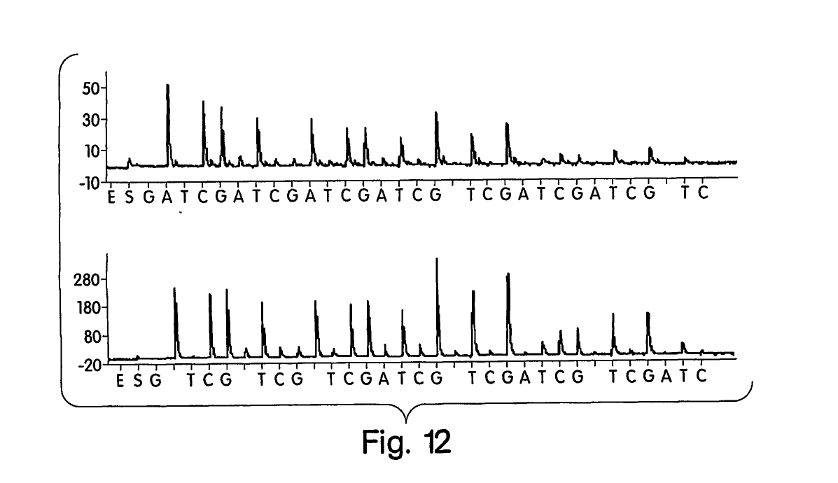

Sensitivity of convective sequencing. FIG. 12 shows a pyrogram (a measurement of photons generated as a consequence of pyrophosphate produced) for the oligo seq1 immobilized onto the Sepharose beads. The number of oligo copies per bead was about 1000. The experimental conditions were the same as described in FIG. 13. From FIG. 14 it is estimated that the signal for the 0.1 uM (ppi) was about 300 counts. The signal for one base (nucleotide A) was ˜38 counts at a copy number of 1000 DNAs per bead. This high sensitivity for convective sequencing is in part due to retention of soluble enzyme activity; since the enzyme mixtures are not immobilized to the beads but are physically trapped in the microstructure of the ultra filtration membrane. Also, the enzyme concentration on the membrane may be increased, resulting in enhanced sensitivity.

It is noted that while in this embodiment two membranes were used (in contact with one another), a single membrane would be equally satisfactory, and it would be routine for the ordinarily skilled artisan to trap the nucleic acids to be sequenced and the necessary sequencing enzymes and substrates on a single membrane.

Effect of immobilization of the luciferase and ATP sulfurylase on Sepharose beads. Sepharose beads are 30±10 μm and have binding capacity of 1×10⁹ biotins per bead. Hence one bead can accommodate the immobilization of luciferase and ATP sulfurylase after the bead becomes loaded with 10⁴-10⁶ copies of nucleic acid template. The effect of immobilization of luciferase and atp sulfurylase on the Sepharose beads with DNA molecules has been studied. 0.5 μl of the oligo seq 1 was added to 100 μl of sepharose beads with prb1 primer. The mixture was placed in a PCR thermocyler and heated to 95° C and allowed to cool to 4° C at rate 0.1° C. s⁻¹. The excess oligo was removed by washing the beads twice with annealing buffer. a 100 μl mixture of luciferase and atp sulfurylase was added to 5 μl of beads and incubate with rotation at 4° C for 1 hr. The pyrogram for the Sepharose beads with the enzyme mixture showed about 3.5 times more sensitivity than the beads without enzymes immobilized to them.

### Example 2

Production of a UMRA

An ultrafiltration membrane is used to create a dense 2-D array of chemical reactions (“unconfined membrane reactor array” or UMRA) in which reactions are seeded by filtering a catalyst or reactant or enzyme onto the filter surface and whereby convective flow washes away laterally diffusing molecules before they contaminate adjacent reactions. Concentration polarization is necessary to create the packed columns of molecules for the UMRA followed by the sequential packing of molecules via concentration polarization to create stacked columns. Reagents are then flowed through a packed column of the UMRA for sequential processing of chemicals. The ultrafiltration membrane may then be bonded to a second, more porous membrane to provide mechanical support to the molecules concentrated by concentration polarization. The membrane is a Molecular/Por membrane (Spectrum Labs) or Anopore™ and Anodisc™ families of ultrafiltration membranes sold, for example, by Whatman PLC.

The substrate material is preferably made of a material that facilitates detection of the reaction event. For example, in a typical sequencing reaction, binding of a dNTP to a sample nucleic acid to be sequenced can be monitored by detection of photons generated by enzyme action on phosphate liberated in the sequencing reaction. Thus, having the substrate material made of a transparent or light conductive material facilitates detection of the photons. Reagents, such as enzymes and template, are delivered to the reaction site by a mobile solid support such as a bead.

The UMRA has handling properties similar to a nylon membrane. Reaction chambers are formed directly on the membrane, such that each reaction site is formed by the woven fibers of the membrane itself. Alternatively, a fiber optic bundle is utilized for the surface of the UMRA. The surface itself is cavitated by treating the termini of a bundle of fibers, e.g., with acid, to form an indentation in the Fiber optic material. Thus, cavities are formed from a fiber optic bundle, preferably cavities are formed by etching one end of the fiber optic bundle. Each cavitated surface can form a reaction chamber, or fiber optic reactor array (FORA). The indentation ranges in depth from approximately one-half the diameter of an individual optical fiber up to two to three times the diameter of the fiber. Cavities are introduced into the termini of the fibers by placing one side of the optical fiber wafer into an acid bath for a variable amount of time. The amount of time varies depending upon the overall depth of the reaction cavity desired (see e.g., Walt, et al., 1996. Anal. Chem. 70: 1888). The opposing side of the optical fiber wafer (i.e., the non-etched side) is typically highly polished so as to allow optical-coupling (e.g., by immersion oil or other optical coupling fluids) to a second, optical fiber bundle. This second optical fiber bundle exactly matches the diameter of the optical wafer containing the reaction chambers, and acts as a conduit for the transmission of light product to the attached detection device, such as a CCD imaging system or camera. The fiber optic wafer is then thoroughly cleaned, e.g. by serial washes in 15% H₂O₂/15%NH₄OH volume:volume in aqueous solution, followed by six deionized water rinses, then 0.5M EDTA, six deionized water washes, 15% H₂O₂/15% NH₄OH and six deionized water washes(one-half hour incubations in each wash).

References

Anderson J R, Chiu D T, Jackman R J, Cherniavskaya O, McDonald J C, Wu H, Whitesides S H, and Whitesides G M. 2000. Fabrication of topologically complex three-dimensional microfluidic systems in PDMS by rapid prototyping. Anal. Chem. 72:3158-64.

Aoki A. 1992. Microwell cluster for processing electron microscope sections for immunocytochemistry. Biotech. Histochem. 67:98-9.

Asoh, H, Nishio, K, Nakao, M. Tamamura, T and Masuda, H. 2001. Conditions for fabrication of ideally ordered anodic porous alumina using pretextured aluminum. J. Electrochem. Soc. 148:B152-B156.

Bickerstaff G F. 1997. Immobilization of Enzymes and Cells. Totowa, N.J., USA: Humana Press.

Cherukuri S C, Demers R R, Fan Z H H, Levine A W, McBride S E, Zanzucchi P J. 1999. Method and system for inhibiting cross-contamination in fluids of combinatorial chemistry device. U.S. Pat. No. 5,980,704.

Crank J. 1975. The Mathematics of Diffusion. 2^{nd} ed. London: Oxford Univ. Press.

Cussler E L. 1997. Diffusion: Mass Transfer in Fluid Systems. 2^{nd} ed. New York: Cambridge Univ. Press.

Dannoux T, Pujol G, and Root D. 2000. High-density test plate and process of making. U.S. Pat. No. 6,030,829.

Deng T, Wu H, Brittain S T, and Whitesides G M. 2000. Prototyping of masks, masters, and stamps/molds for soft lithography using an office printer and photographic reduction. Anal. Chem. 72:3176-80.

Ehrfeld, W, Hessel, V, and Löwe, H. 2000. Microreactors: New Technology for Modern Chemistry. New York: Wiley-VCH.

Eykamp, W. 1995. Microfiltration and ultrafiltration, pp. 143 in Membrane Separation Technology: Principles and Applications, R D Noble and S A Stern, eds. Amsterdam: Elsevier Science BV.

Ferguson J A, Boles C, Adams C P, and Walt D R. 1996. A fiber-optic DNA biosensor microarray for the analysis of gene expression. Nature Biotech. 14:1681-4.

Furneaux, R C, Rigby, W R, and Davidson, A P. 1989. The formation of controlled porosity membranes from anodically oxidized aluminum. Nature. 337:147-9.

Gamble R C, Theriault T P, Baldeschwieler J D. 1999. Apparatus for the chemical synthesis of molecular arrays. U.S. Pat. No. 5,981,733.Hughes T R, Mao, M, Jones, A R, Burchard J., Marton M J, Shannon K W, Lefkowitz S M, Ziman M, Schelter J M, Meyer M R, Kobayashi S, Davis C, Dai H Y, He Y D D, Stephaniants S B, Cavet G, Walker W L, West A, Coffey E, Shoemaker D D, Stoughton R, Blanchard A P, Friend S H, and Linsley P S. 2001. Expression profiling using microarrays fabricated by an ink-jet oligonucleotide synthesizer. Nat. Biotechnol. 19(4):342-347.

Hyman E D. 1988. A new method for sequencing DNA. Anal. Biochem. 174:423-36.

Jansson V and Jansson K. 2002. Enzymatic chemiluminescence assay for inorganic pyrophosphate. Analytical Biochemistry. 304: 135-137.

Jensen N. 2002. Flow-through processing on a microchip for DNA pyrosequencing. Natl. Nanofabrication Users Network 66-67.

Kane R S, Takayama S, Ostuni E, Ingber D E, and Whitesides G M. 1999. Patterning proteins and cells using soft lithography. Biomaterials. 20:2363-76.

Kulkarni, S S, Funk, E W, and Li, N N. 1992. Ultrafiltration, pp. 391-453 in Membrane Handbook, W S W Ho and K K Sirkar, eds. New York: Van Nostrand Reinhold.

Lehninger, A L. 1975. Biochemistry, 2^{nd} ed. New York: Worth Publishers.

Lonsdale, H K. 1982. The growth of membrane technology. J. Memb. Sci. 10:81-181.

Madou M J 1997. Fundamentals of Microfabrication. Boca Raton, Fla., USA: CRC Press.

Mardilovich, P P, Govyadinov, A N, Mukhurov, N I, Rzhevskii, A M, and Paterson, R. New and modified anodic alumina membranes. Part 1: Treatment of anodic alumina membranes. Part II: Comparison of solubility of amorphous (normal) and polycrystalline anodic alumina membranes. J. Memb. Sci. 98:131-155.

Martin C R. 1994. Nanomaterials—A membrane-based synthetic approach. Science. 266:1961-1966.

Matsuda T and Chung D J. 1994. Microfabricated surface designs for cell culture and diagnosis. ASAIO J. 1:M594-7.

Michael K L, Taylor L C, Schultz S L, and Walt D R. 1998. Randomly ordered addressable high-density optical sensor arrays. Anal. Chem. 70:1242-8.

Mulder, M H V. 1995. Polarization phenomena and membrane fouling, pp.45-84 in Membrane Separation Technology: Principles and Applications, R D Noble and S A Stern, eds. Amsterdam: Elsevier Science BV.

Nyrén P, Pettersson B, and Uhlén M. 1993. Solid phase DNA minisequencing by an enzymatic luminometric inorganic pyrophosphate detection assay. Anal. Biochem. 208:171-5.

Rai-Choudhury P. 1997. Handbook of Microlithography, Micromachining and Microfabrication. Vol. 1: Microlithography and Vol. 2: Micromachining and Microfabrication. SPIE Press.

Ronaghi M, Uhlën M, and Nyrén P. 1998. A sequencing method based on real-time pyrophosphate. Science. 281:363-5.

Schuller J. 2002. Microfluidics for DNA pyrosequencing. Natl. Nanofabrication Users Network. 76-77.

Tanford, C. 1961. Physical Chemistry of Macromolecules. New York: Wiley. Taylor L C and Walt D R. 2000. Application of high-density optical microwell arrays in a live-cell biosensing system. Anal. Biochem. 278:132-42

Zhu H, Klemic J F, Chang S, Bertone P, Casamayor A, Klemic K G, Smith D, Gerstein M, Reed M A, and & Snyder M. 2000. Analysis of yeast protein kinases using protein chips. Nature Genet. 26:283-89.

1. A CMRA comprising:
(a) a microreactor element comprising an array of open microchannels or open microwells, the longitudinal axes of said microchannels or microwells arranged in a substantially parallel manner; and
(b) a porous filter element in contact with the microreactor element to form a bottom to the microchannels or microwells, thereby defining a series of reaction chambers, wherein the porous filter element comprises a permselective membrane that blocks the passage of nucleic acids, proteins and beads there across, but permits the passage of low molecular weight solutes, organic solvents and water there across.

2. The CMRA of claim 1, wherein the microreactor element comprises a plate formed from a fused fiber optic bundle, wherein the microchannels extend from the top face of the plate through to the bottom face of the plate.

3. The CMRA of claim 1 further comprising an additional porous support between the microreactor element and the porous filter element.

4. The CMRA of claim 1 wherein the porous filter element comprises an ultrafilter.

5. The CMRA of claim 1 further comprising at least one mobile solid support disposed in each of a plurality of the microchannels of the microreactor element.

6. The CMRA of claim 5 wherein the mobile solid support is a bead.

7. The CMRA of claim 1 or claim 6 wherein the mobile solid support has an enzyme and/or a nucleic acid immobilized thereon.

8. A method of making the CMRA of claim 1 comprising attaching a microreactor element to a porous filter element.

9. A UMRA comprising a porous filter element against which molecules are concentrated by concentration polarization wherein discrete reaction chambers are formed in discrete locations on the surface of or within the porous filter element by depositing reactant molecules at discrete sites on or within the porous filter element.

10. The UMRA of claim 9 wherein the reaction chambers are formed by depositing mobile solid supports having said reactant molecules immobilized thereon, on the surface of, or within, the porous element.

11. The UMRA of claim 9 wherein the porous filter element comprises an ultrafilter.

12. The UMRA of claim 9 wherein the mobile solid support is a bead.

13. The UMRA of claim 10 or claim 12 wherein the mobile solid support has an enzyme and/or a nucleic acid immobilized thereon.

14. A UMRA comprising:
(a) a porous membrane with discrete reaction sites formed by depositing mobile solid supports having said reactant molecules thereon, on the surface of, or within the porous membrane;
(b) a nucleic acid template immobilized to a solid support; and
(c) optionally, at least one immobilized enzyme.

15. The UMRA of claim 14 wherein the mobile solid support is a bead.

16. The UMRA of claim 14 wherein the porous membrane is nylon membrane.

17. The UMRA of claim 14 wherein the porous membrane is made of a woven fiber.

18. The UMRA of claim 14 wherein the porous membrane pore size at least 0.02 μm.

19. The UMRA of claim 1 wherein the solid support is selected from the group consisting of a bead, glass surface, fiber optic or the porous membrane.

20. The UMRA of claim 14 wherein the immobilized enzyme is immobilized to a bead or the porous membrane.

21. The UMRA of claim 14 wherein the immobilized enzyme is selected from the group consisting of ATP sulfurylase, luciferase, hypoxanthine phosphoribosyltransferase, xanthine oxidase, uricase or peroxidase.

22. An array comprising:
(a) a first porous membrane with a plurality of discrete reaction sites disposed thereon, and/or within, wherein each reaction site has immobilized template adhered to the surface; and
(b) a second porous membrane with at least one enzyme located on the surface of, and/or within, the membrane, wherein the second porous membrane is in direct contact with the first porous membrane.

23. The array of claim 22 wherein the first or second membrane is a nylon membrane.

24. The array of claim 22 wherein the porous membrane is made of a woven fiber.

25. The array of claim 22 wherein the each reaction site is defined by the pores of the porous membrane.

26. The array of claim 22 wherein the first and second porous membranes have a pore size of at least 0.2 μm.

27. The array of claim 22 wherein the template is immobilized to a bead or to the porous membrane.

28. The array of claim 22 wherein the enzyme is selected from the group consisting of ATP sulfurylase, luciferase, hypoxanthine phosphoribosyltransferase, xanthine oxidase, uricase or peroxidase.

29. A CMRA comprising an array of open microchannels or microwells attached to a porous filter or membrane.

30. The CMRA of claim 29 further comprising a mechanical support, wherein the mechanical support separates the microchannels from the porous membrane.

31. The CMRA of claim 30 wherein the mechanical support is selected from the group consisting of plastic mesh, wire screening or molded or machined spacers.

32. The CMRA of claim 29 wherein the porous membrane is a nylon membrane.

33. The CMRA of claim 29 wherein the porous membrane is made of a woven fiber.

34. The CMRA of claim 29 wherein the membrane pore size is at least 0.02 μm.

35. The CMRA of claim 29 wherein the microchannels are formed by concentration polarization.

36. An apparatus for determining the nucleic acid sequence in a template nucleic acid polymer, comprising:
(a) a CMRA or UMRA;
(b) nucleic acid delivery means for introducing template nucleic acid polymers to the discrete reaction sites;
(c) nucleic acid delivery means to deliver reagents to the reaction sites to create a polymerization environment in which the nucleic acid polymers will act as template polymers for the synthesis of complementary nucleic acid polymers when nucleotides are added;
(d) convective flow delivery means to immobilize reagents to the porous membrane;
(e) detection means for detecting the formation of inorganic pyrophosphate enzymatically; and
(f) data processing means to determine the identity of each nucleotide in the complementary polymers and thus the sequence of the template polymers.

37. The apparatus of claim 36 wherein the porous membrane is a nylon membrane.

38. The apparatus of claim 36 wherein the nylon membrane is made of a woven fiber.

39. The apparatus of claim 36 wherein the pore size is at least 0.02 μm.

40. The apparatus of claim 36 wherein the discrete reaction sites are formed by concentration polarization.

41. The apparatus of claim 36 wherein the convective flow delivery means is a syringe or a peristaltic pump.

42. The apparatus of claim 36 wherein the template nucleic acid is attached to a solid support.

43. The apparatus of claim 42 wherein the solid support is selected from the group consisting of a bead, glass surface, fiber optic or porous membrane.

44. The apparatus of claim 36 wherein the enzyme detecting inorganic pyrophosphate is selected from the group consisting of ATP sulfurylase, luciferase, hypoxanthine phosphoribosyltransferase, xanthine oxidase, uricase or peroxidase.

45. The apparatus of claim 36 wherein the detection means is a CCD camera.

46. The apparatus of claim 36 wherein the data processing means is a computer.

47. An apparatus for processing a plurality of analytes, the apparatus comprising:
(a) a CMRA or an UMRA;
(b) fluid means for delivering processing reagents from one or more reservoirs to the flow chamber so that the analytes disposed therein are exposed to the reagents; and
(c) detection means for detecting a sequence of optical signals from each of the reaction sites, each optical signal of the sequence being indicative of an interaction between a processing reagent and the analyte disposed in the reaction site, wherein the detection means is in communication with the reaction site.

48. The apparatus of claim 47 wherein the porous membrane is a nylon membrane.

49. The apparatus of claim 47 wherein the pore size is at least 0.02 μm.

50. The apparatus of claim 47 wherein the template nucleic acid is attached to a solid support.

51. The apparatus of claim 50 wherein the solid support is selected from the group consisting of a bead, glass surface, fiber optic or porous membrane.

52. The apparatus of claim 47 wherein the convective flow delivery means is a peristaltic pump.

53. The apparatus of claim 47 wherein the enzyme detecting inorganic pyrophosphate is selected from the group consisting of ATP sulfurylase, luciferase, hypoxanthine phosphoribosyltransferase, xanthine oxidase, uricase or peroxidase.

54. The apparatus of claim 47 wherein the detection means is a CCD camera.

55. The apparatus of claim 47 wherein the data processing means is a computer.

56. An apparatus for determining the base sequence of a plurality of nucleotides on an array, the apparatus comprising:
(a) a CMRA or UMRA;
(b) reagent delivery means for adding an activated nucleotide 5′-triphosphate precursor of one known nitrogenous base to a reaction mixture to each reaction site, each reaction mixture comprising a template-directed nucleotide polymerase and a single-stranded polynucleotide template hybridized to a complementary oligonucleotide primer strand at least one nucleotide residue shorter than the templates to form at least one unpaired nucleotide residue in each template at the 3′-end of the primer strand, under reaction conditions which allow incorporation of the activated nucleoside 5′-triphosphate precursor onto the 3′-end of the primer strands, provided the nitrogenous base of the activated nucleoside 5′-triphosphate precursor is complementary to the nitrogenous base of the unpaired nucleotide residue of the templates;
(c) detection means for detecting whether or not the nucleoside 5′-triphosphate precursor was incorporated into the primer strands in which incorporation of the nucleoside 5′-triphosphate precursor indicates that the unpaired nucleotide residue of the template has a nitrogenous base composition that is complementary to that of the incorporated nucleoside 5′-triphosphate precursor; and
(d) means for sequentially repeating steps (b) and (c), wherein each sequential repetition adds and detects the incorporation of one type of activated nucleoside 5′-triphosphate precursor of known nitrogenous base composition; and
(e) data processing means for determining the base sequence of the unpaired nucleotide residues of the template in each reaction chamber from the sequence of incorporation of said nucleoside precursors.

57. The apparatus of claim 56 wherein the porous membrane is a nylon membrane.

58. The apparatus of claim 53 wherein the pore size is at least 0.02 μm.

59. The apparatus of claim 53 wherein the template nucleic acid is attached to a solid support.

60. The apparatus of claim 59 wherein the solid support is selected from the group consisting of a bead, glass surface, fiber optic or porous membrane.

61. The apparatus of claim 53 wherein the convective flow delivery means is a peristaltic pump.

62. The apparatus of claim 53 wherein the enzyme detecting inorganic pyrophosphate is selected from the group consisting of ATP sulfurylase, luciferase, hypoxanthine phosphoribosyltransferase, xanthine oxidase, uricase or peroxidase.

63. The apparatus of claim 53 wherein the detection means is a CCD camera.

64. The apparatus of claim 53 wherein the data processing means is a computer.

65. An apparatus for determining the nucleic acid sequence in a template nucleic acid polymer, comprising:
(a) a CMRA or UMRA;
(b) nucleic acid delivery means for introducing a template nucleic acid polymers onto the reaction sites;
(c) nucleic acid delivery means to deliver reagents to the reaction chambers to create polymerization environment in which the nucleic acid polymers will act as a template polymers for the synthesis of complementary nucleic acid polymers when nucleotides are added;
(d) reagent delivery means for successively providing to the polymerization environment a series of feedstocks, each feedstock comprising a nucleotide selected from among the nucleotides from which the complementary nucleic acid polymer will be formed, such that if the nucleotide in the feedstock is complementary to the next nucleotide in the template polymer to be sequenced said nucleotide will be incorporated into the complementary polymer and inorganic pyrophosphate will be released;
(e) detection means for detecting the formation of inorganic pyrophosphate enzymatically; and
(f) data processing means to determine the identity of each nucleotide in the complementary polymers and thus the sequence of the template polymers.

66. The apparatus of claim 65 wherein the porous membrane is a nylon membrane.

67. The apparatus of claim 65 wherein the pore size is at least 0.02 μm.

68. The apparatus of claim 65 wherein the template nucleic acid is attached to a solid support.

69. The apparatus of claim 68 wherein the solid Support is selected from the group consisting of a bead, glass surface, fiber optic or porous membrane.

70. The apparatus of claim 65 wherein the convective flow delivery means is a peristaltic pump.

71. The apparatus of claim 65 wherein the enzyme detecting inorganic pyrophosphate is selected from the group consisting of ATP sulfurylase, luciferase, hypoxanthine phosphoribosyltransferase, xanthine oxidase, uricase or peroxidase.

72. The apparatus of claim 65 wherein the detection means is a CCD camera.

73. The apparatus of claim 65 wherein the data processing means is a computer.

74. A system for sequencing a nucleic acid comprising the following components:
(a) a CMRA or UMRA;
(b) at least one enzyme immobilized on a solid support;
(c) means for flowing reagents over said porous membrane;
(d) means for detection; and
(e) means for determining the sequence of the nucleic acid.

75. The system of claim 74 wherein the porous membrane is a nylon membrane.

76. The system of claim 74 wherein the porous membrane has a pore size is at least 0.02 μm.

77. The system of claim 74 wherein the reaction sites are formed by concentration polarization.

78. The system of claim 74 wherein the immobilized enzyme is selected from the group consisting of ATP sulfurylase, luciferase, hypoxanthine phosphoribosyltransferase, xanthine oxidase, uricase or peroxidase.

79. The system of claim 74 wherein the solid support is selected from the group consisting of a bead, glass surface, fiber optic or porous membrane.

80. The system of claim 74 wherein the means for detection is a CCD camera.

81. The system of claim 74 wherein the means for determining a sequence is by pyrophosphate sequencing.

82. A system for sequencing a nucleic acid comprising the following components:
(a) a CMRA or UMRA
(b) at least one enzyme immobilized on a solid support;
(c) means for flowing reagents over said porous membrane;
(d) means for enzymatic detection; and
(e) means for determining the sequence of the nucleic acid.

83. The system of claim 82 wherein the porous membrane is a nylon membrane.

84. The system of claim 82 wherein the porous membrane has a pore size is at least 0.02 μm.

85. The system of claim 82 wherein the reaction sites are formed by concentration polarization.

86. The system of claim 82 wherein the immobilized enzyme is selected from the group consisting of ATP sulfurylase, luciferase, hypoxanthine phosphoribosyltransferase, xanthine oxidase, uricase or peroxidase.

87. The system of claim 82 wherein the solid support is selected from the group consisting of a bead, glass surface, fiber optic or porous membrane.

88. The system of claim 82 wherein the means for detection is a CCD camera.

89. The system of claim 82 wherein the means for determining a sequence is by pyrophosphate sequencing.

90. A method for carrying out separate parallel independent reactions in an aqueous environment, comprising:
(a) delivering a fluid containing at least one reagent to an array, using the CMRA of claim 1 or the UMRA of claim 9, wherein each of the reaction sites immersed in a substance such that when the fluid is delivered onto each reaction site, the fluid does not diffuse onto an adjacent site;
(b) washing the fluid from the array in the time period after the starting material has reacted with the reagent to form a product in each reaction site;
(c) sequentially repeating steps (a) and (b).

91. The method of claim 90 wherein the product formed in any one reaction chamber is independent of the product formed in any other reaction chamber, but is generated using one or more common reagents.

92. The method of claim 90 wherein the starting material is a nucleic acid sequence and at least one reagent in the fluid is a nucleotide or nucleotide analog.

93. The method of claim 90 wherein the fluid additionally comprises a polymerase capable of reacting the nucleic acid sequence and the nucleotide or nucleotide analog.

94. The method of claim 90 additionally comprising repeating steps (a) and (b) sequentially.

95. The method of claim 90 wherein the substance is mineral oil.

96. The method of claim 90 wherein the reaction sites are defined by concentration polarization.

97. A method of determining the base sequence of nucleotides in an array format, the method comprising the steps of:
(a) adding an activated nucleoside 5′-triphopsphate precursor of one known nitrogenous base composition to a plurality of reaction sites localized on a CMRA or UMRA, wherein the reaction site is comprised of a template-directed nucleotide polymerase and a heterogenous population of single stranded templates hybridized to complementary oligonucleotide primer strands at least one nucleotide residue shorter than the templates to form at least one unpaired nucleotide residue in each template at the 3′ end of the primer strand under reaction conditions which allow incorporation of the activated nucleoside 5′-triphosphate precursor onto the 3′ end of the primer strand under reaction conditions which allow incorporation of the activated nucleoside 5′-triphosphate precursor onto the 3′ end of the primer strands, provided the nitrogenous base of the activated nucleoside 5′-triphosphate precursor is complementary to the nitrogenous base of the unpaired nucleotide residue of the templates;
(b) detecting whether or not the nucleoside 5′-triphosphate precursor was incorporated into the primer strands in which incorporation of the nucleoside 5′-triphosphate precursor indicates that the unpaired nucleotide residue of the template has a nitrogenous base composition that is complementary to that of the incorporated nucleoside 5′-triphosphate precursor; and
(c) sequentially repeating steps (a) and (b), wherein each sequential repetition adds and detects the incorporation of one type of activated nucleoside 5′-triphosphate precursor of known nitrogenous base composition;
(d) determining the base sequence of the unpaired nucleotide residues of the template from the sequence of incorporation of said nucleoside precursors.

98. The method of claim 97 wherein the detection of the incorporation of the activated precursor is accomplished enzymatically.

99. The method of claim 98 wherein the enzyme is selected from the group consisting of ATP sulfurylase, luciferase, hypoxanthine phosphoribosyltransferase, xanthine oxidase, uricase or peroxidase.

100. The method of claim 97 wherein the enzyme is immobilized to a solid support.

101. The method of claim 97 wherein the solid support is selected from the group comprising a bead, glass surface, fiber optic or porous membrane.

102. A method of determining the base sequence of a plurality of nucleotides on an array, said method comprising:
(a) providing a plurality of sample DNA's, each disposed within a plurality of reaction sites on a CMRA or UMRA;
(b) detecting the light level emitted from a plurality of reaction sites on respective proportional of an optically sensitive device;
(c) converting the light impinging upon each of said portions of said optically sensitive device into an electrical signal which is distinguishable from the signals from all of said other regions;
(d) determining a light intensity for each of said discrete regions from the corresponding electrical signal;
(e) recording the variations of said electrical signals with time.

103. The method of claim 102 wherein the porous membrane is a nylon membrane.

104. The method of claim 102 wherein the pore size is at least 0.2 μm.

105. The method of claim 102 wherein the detection is performed enzymatically.

106. The method of claim 105 wherein the enzyme is selected from the group consisting of ATP sulfurylase, luciferase, hypoxanthine phosphoribosyltransferase, xanthine oxidase, uricase or peroxidase.
